SEQUENCE LISTING (6 sequences)

SEQ ID NO: 1 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 2 - DNA, 1068 bp, Homo sapiens
atggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtaa

SEQ ID NO: 3 - protein, 353 aa, Mus musculus
MDYTMEPNVTMTDYYPDFFTAPCDAEFLLRGSMLYLAILYCVLFVLGLLGNSLVILVLVGCKKLRSITDIYLLNLAASDLLFVLSIPFQTHNLLDQWVFGTAMCKVVSGLYYIGFFSSMFFITLMSVDRYLAIVHAVYAIKVRTASVGTALSLTVWLAAVTATIPLMVFYQVASEDGMLQCFQFYEEQSLRWKLFTHFEINALGLLLPFAILLFCYVRILQQLRGCLNHNRTRAIKLVLTVVIVSLLFWVPFNVALFLTSLHDLHILDGCATRQRLALAIHVTEVISFTHCCVNPVIYAFIGEKFKKHLMDVFQKSCSHIFLYLGRQMPVGALERQLSSNQRSSHSSTLDDIL

SEQ ID NO: 4 - DNA, 1062 bp, Mus musculus
atggattacacgatggagcccaacgtcacgatgaccgactactaccctgatttcttcaccgccccctgtgacgcagagttcctcctcaggggcagcatgctgtatctggccatcttgtactgcgtcttgtttgtgctgggccttctggggaacagcctggtcatcttagtcctcgtgggctgcaagaaactgaggagcatcacagatatctacctcctgaacctggccgcatccgacctgctctttgtcctctctattccttttcagacccacaacctgctggaccagtgggtgtttgggactgcgatgtgtaaggtggtctctggcctttattacattggttttttcagcagtatgttcttcatcaccctaatgagtgtggacaggtatctggctattgtccacgctgtctatgccatcaaggtgaggacggccagcgtgggcacagccctgagtctgacagtgtggctggctgctgtcacagccaccatccccttgatggttttttaccaagtggcctctgaagacggcatgctacaatgtttccagttttatgaagagcagtctttgaggtggaagctctttacccactttgaaatcaacgccttgggtctgctgctcccctttgccatcctcctgttctgctatgtcaggatcctgcagcagctgcggggctgcctgaaccacaacaggaccagagccatcaagctggtgctcaccgtagtcattgtgtctttactcttctgggtcccattcaacgtggcccttttcctcacgtccctgcacgacctgcacatcttggatggatgtgccacgaggcagaggctggctctggccatccatgtcacagaggtcatctcttttacccactgctgcgtgaaccccgtcatctacgcgttcataggagagaagtttaagaaacacctcatggatgtgtttcaaaagagctgcagccacatcttcctctacttagggagacaaatgcccgtgggggcgttggaaaggcagctgtcctcgaaccagcgatcttcccattcttccaccctggatgacatcttgtaa

SEQ ID NO: 5 - DNA, 1557 bp, Artificial Sequence
  anti-EpCAM CAR
atggcctcaccgttgacccgctttctgtcgctgaacctgctgctgctgggtgagtcgattatcctggggagtggagaagctgaggtgcagctggccgaatctggcggcggactggtgcagcccggcagatccatgaagctgagctgcgctgccagcggcttcaccttcagcaacttccccatggcctgggtgcgccaggcccccaccaagtgtctggaatgggtggccaccatcagcaccagcggcggcagcacctactaccgggacagcgtgaagggccggttcaccatcagccgggacaacgccaagagcaccctgtacctgcagatgaacagcctgcggagcgaggacaccgccacctactactgcacccggaccctgtacatcctgcgggtgttctacttcgactactggggccagggcgtgatggtgacagtgtctagcggcggaggcggcagcggaggtggaggaagtggcggcggaggatccgacatccagatgacccagtctcccgccagcctgagcgcctctctgggcgagacagtgtccatcgagtgcctggccagcgagggcatcagcaacgacctggcctggtatcagcagaagtccggcaagagcccccagctgctgatctacgccaccagcagactgcaggacggcgtgcccagcagattcagcggcagcggctccggcacccggtacagcctgaagatcagcggcatgcagcccgaggacgaggccgactacttctgccagcagagctacaagtacccctggaccttcggctgcggcacaaagctggaactgaagggcggagggggctctgggggaggcggatctctcgaggaacagaagctgatcagcgaagaggacctgactactaccaagccagtgctgcgaactccctcacctgtgcaccctaccgggacatctcagccccagagaccagaagattgtcggccccgtggctcagtgaaggggaccggattggacttcgcctgtgatatttacttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 6 - DNA, 627 bp, Artificial Sequence
  dominant-negative TGF-B receptor 2
gccaccatgggtcgggggctgctcaggggcctgtggccgctgcacatcgtcctgtggacgcgtatcgccagcacgatcccaccgtatgatgttcctgattatgctagcctccacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacgctagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgtag

(19) United States
(12) Patent Application Publication
(10) Pub. No.: US 2022/0143087 A1
(43) Pub. Date: May 12, 2022

(54) CCR8 EXPRESSING LYMPHOCYTES FOR TARGETED TUMOR THERAPY

(71) Applicant: Klinikum der Universitat Munchen, Munchen (DE)

(72) Inventors: Sebastian Kobold, Muenchen (DE); Stefan Endres, Munchen (DE); Bruno Cadilha, Munchen (DE)

(21) Appl. No.: 17/435,634

(22) PCT Filed: Mar. 6, 2020
(86) PCT No.: PCT/EP2020/056086

§ 371 Date: Sep. 1, 2021

(30) Foreign Application Priority Data
Mar. 8, 2019 (EP) 19161708.3

(51) Int. Cl. A61K 35/17; C12N 5/0783

(52) U.S. Cl. CPC A61K 35/17; C12N 2510/00; C12N 2501/515; C12N 5/0636

(57) ABSTRACT

The present invention relates to lymphocytes genetically engineered to express a CCR8 polypeptide or a functional variant thereof for use in targeted tumor immunotherapies such as adoptive T cell therapy.

### 1. BACKGROUND

The present invention relates to lymphocytes genetically engineered to express a CCR8 polypeptide or a functional variant thereof for use in targeted tumor immunotherapies such as adoptive T cell therapy, as well as nucleic acids, vectors and methods of use in the production of such cells as well as kits comprising such cells, nucleic acids and/or vectors. The cells of the invention are preferably human lymphocytes and more preferably primary human lymphocytes such as NK cells or T cells, including CD3+ T cells, CD8+ T cells, CD4+ T cells, and γδ T cells. Most preferably, the cells of the invention are primary human T cells. The invention provides the lymphocytes genetically engineered to express CCR8 or a functional variant thereof as well as pharmaceutical compositions comprising such lymphocytes for use in a method of treatment of diseases characterized by the expression of CCL1 within the diseased parenchyma (or parenchyma associated with the disease), e.g., for use in a method of treatment of cancer characterized by the expression of CCL1 within the cancer parenchyma.

The use of engineered immune cells in therapy has been demonstrated, in particular, in with adoptive T cell therapy (ACT) for the treatment of cancers. ACT is a powerful treatment approach using, in this context, cancer-specific T cells (Rosenberg and Restifo, Science 348(2015), 62-68). ACT may use naturally occurring tumor-specific cells or T cells rendered specific by genetic engineering, e.g., to express recombinant T cell or chimeric antigen receptors (Rosenberg and Restifo, Science 348(2015), 62-68). ACT has been demonstrated to successfully treat and induce remission in patients suffering from advanced and otherwise treatment refractory diseases such as acute lymphatic leukemia, non-Hodgkin's lymphoma or melanoma (Dudley et al., J Clin Oncol 26(2008), 5233-5239; Grupp et al., N Engl J Med 368 (2013), 1509-1518; Kochenderfer et al., J Clin Oncol. 33(2015), 540-5499; Maude et al., N Engl J Med 378(2018), 439-448; Schuster et al., N Engl J Med 380(2019), 45-56). However, long term benefits are restricted to a small subset of patients, with the remaining eventually relapsing and succumbing to their refractory disease.

An element believed essential for the success of ACT is T cell access to the tumor cells or tissue. Therefore strategies enabling T cell entry need to be developed and implemented (Gattinoni et al., Nat Rev Immunol 6(2006), 383-393). Currently, the most effective method to of enhancing T cell infiltration is total body irradiation prior to ACT. Such irradiation permeabilizes tumor tissue, remodels the vasculature and depletes suppressive cells (Dudley et al., J Clin Oncol 23(2005), 2346-2357). While this strategy has shown efficacy in clinical trials, its non-specific nature induces severe side effects, limiting its applicability and highlighting the need for more focused strategies (Dudley et al., J Clin Oncol 23(2005), 2346-2357).

T cell entry and trafficking into tissues is a tightly regulated process wherein integrins and chemokines play a central role (Franciszkiewicz et al., Cancer Res 72(2012), 6325-6332; Kalos and June, Immunity 39(2013), 49-60). Chemokines are secreted by resident cells, forming gradients in vivo that not only attract cells bearing the corresponding receptor but that also regulate tissue penetration (Franciszkiewicz et al., Cancer Res 72(2012), 6325-6332). With respect to tumors, characterizing chemokines can be expressed and secreted by the tumor cells themselves or may be expressed and secreted by cells associated with the tumor parenchyma, e.g., infiltrating immune cells. Tumors and tumor parenchyma have been demonstrated to express advantageous chemokine profiles, e.g., that attract immune suppressive cell populations and/or excluding proinflammatory subsets (Curiel et al., Nat Med 10(2004), 942-949).

Introducing receptors specific for chemokines expressed within tumor tissue into T cells has been demonstrated to redirect and enhance antigen-specific migration of the T cells to and into the tumor tissue. Such receptors already tested in preclinical models include CCR2, CCR4 and CXCR2. Although apparently enhancing the targeting and/or specificity of ACT, the therapy generally failed to reject tumors, indicating insufficient infiltration and functionality at the tumor site (Di Stasi et al., Blood 113(2009), 6392-6402; Peng et al., Clin Cancer Res 16(2010), 5458-5468; Asai et al., PLoS One 8(2013), e56820). Accordingly, there is remains a need in the art to improve targeted tumor therapy, e.g., ACT. Such improvements include tools having the potential to improve safety and efficacy of the ACT, in particular, to overcome the above disadvantages.

### 2. SUMMARY

The present invention provides a lymphocyte genetically engineered to express a chemokine receptor 8 polypeptide (CCR8) or a functional variant thereof. The CCR8 polypeptide is preferably human CCR8 as known in the art, e.g., having SEQ ID NO:1, as defined in UniProt P51685 (https://www.uniprot.org/uniprot/P51685). An exemplary nucleic acid sequence encoding CCR8 is SEQ ID NO:2. A functional variant of CCR8 is a polypeptide that does not have an amino acid sequence identical to SEQ ID NO:1, but which polypeptide exhibits or imparts the same functional activity as CCR8 when expressed by the lymphocyte, i.e., the polypeptide (functional variant) is characterized by CCR8 activity. The functional variant can be an amino acid sequence variant polypeptide having an amino acid sequence that is at least 85%, 86%, 87%, 88%, 89%, 90%, 91%, 92%, 93%, 94%, 95%, 96%, 97%, 98% or 99% identical to SEQ ID NO:1 provided that the sequence variant is characterized by CCR8 activity. The functional variant can also be a fragment of CCR8 (SEQ ID NO:1) or a fragment of the amino acid sequence variant as described in this paragraph, provided that the fragment is characterized by CCR8 activity. Accordingly, a functional variant of CCR8 can be (i) an amino acid sequence variant of SEQ ID NO:1, e.g., having at least 85% sequence identity to SEQ ID NO:1, (ii) can be a fragment of SEQ ID NO:1, or (iii) can be a fragment of the amino acid sequence variant of SEQ ID NO:1 provided that the amino acid sequence variant, the fragment, or the variant fragment is characterized by CCR8 activity. Therefore, the invention provides a lymphocyte genetically engineered to express CCR8 polypeptide having the amino acid sequence of SEQ ID NO:1, or a functional variant thereof (i.e., an amino acid sequence variant CCR8 polypeptide having an amino acid sequence at least 85% identical to SEQ ID NO:1 characterized by CCR8 activity, a fragment of SEQ ID NO:1, or an amino acid sequence variant of the fragment provided the fragment or its variant is characterized by CCR8 activity).

The lymphocyte of the invention has been genetically engineered to express CCR8 or a functional variant thereof. Accordingly, it is understood that the lymphocyte does not express CCR8 or the functional variant endogenously, but has been genetically engineered so as to comprise at least one of the following polynucleotides: (i) an exogenous polynucleotide encoding a polypeptide having the amino acid sequence of CCR8 (SEQ ID NO:1); (ii) a polynucleotide encoding an amino acid variant polypeptide of CCR8, having an amino acid sequence at least 85% identical to SEQ ID NO:1 and further characterized by having CCR8 activity; (iii) a polynucleotide encoding a fragment of the polypeptide encoded by the polynucleotide of (i) or (ii) and further characterized by having CCR8 activity; (iv) a polynucleotide comprising the nucleic acid sequence SEQ ID NO:2; or (v) a polynucleotide sequence having at least 85% sequence identity to SEQ ID NO:2 and encoding a polypeptide having CCR8 activity.

The invention also provides methods to produce a lymphocyte genetically engineered to express a CCR8 polypeptide (SEQ ID NO:1) or a functional variant thereof (i.e., an amino acid sequence variant of CCR8 having at least 85% sequence identity and characterized in having CCR8 activity; or a fragment of CCR8 (SEQ ID NO:1) or its amino acid sequence variant wherein the fragment is characterized by having CCR8 activity) comprising

(a) introducing into the lymphocyte
(i) an exogenous polynucleotide encoding SEQ ID NO:1;
(ii) an exogenous polynucleotide encoding a functional variant of SEQ ID NO:1, which may be
   (1) a polypeptide having an amino acid sequence at least 85% identical to SEQ ID NO:1 and which is further characterized in having CCR8 activity; or
   (2) a fragment of the polypeptide encoded by polynucleotide of (i) or (ii)(1), which fragment is characterized in having CCR8 activity;
(iii) a polynucleotide comprising or consisting of the nucleic acid sequence of SEQ ID NO:2: or
(iv) a polynucleotide comprising or consisting of a nucleic acid sequence having at least 85% sequence identity to SEQ ID NO:2 that encodes a polypeptide characterized in having CCR8 activity;
   (b) culturing the lymphocyte engineered according to (a) under conditions allowing the expression of the CCR8 polypeptide, the amino acid sequence variant CCR8 polypeptide or fragment of either; and (c) recovering the engineered lymphocyte.

The invention is directed to lymphocytes genetically engineered to express CCR8 (SEQ ID NO:1), and/or variants thereof characterized by having CCR8 activity, specifically, an amino acid sequence variant of CCR8 having at least 85% sequence identity to SEQ ID NO:1, a fragment of SEQ ID NO:1, or a fragment of the amino acid sequence variant, wherein the amino acid sequence variants and the fragments are characterized as having CCR8 activity. It will be appreciated that the polypeptides characterized as having CCR8 activity are polypeptides that impart CCR8 activity to the lymphocyte genetically engineered to express them. The CCR8 activity of the cells, and, thus, of the polypeptide (i.e., amino acid sequence variants of CCR8 and/or fragments as described herein), can be assessed by any means known in the art and/or described herein. Non-limiting examples of method to assess CCR8 activity include chemotactic and/or migration assays mediated by the CCR8 ligand, CCL1; and assessment of CCL1 induced binding to ICAM-1.

The genetically engineered lymphocytes and methods of their production and use are provided not only as tools for the treatment of disease (i.e., are provided not only as therapeutic tools such as a medicament) but will be also be understood to have applicability as model systems for investigating disease therapies. Accordingly, while the genetically engineered lymphocytes of the invention as disclosed herein are preferably human lymphocytes, more preferably primary human lymphocytes (e.g., including NK cells and T cells), and most preferably primary human T cells (e.g., including CD3+ T cells, CD4+ T cells, CD8+ T cells, γδ T cells, invariant T cells and NK T cells), the invention also encompasses genetically engineered lymphocytes that are derived from lymphocyte cell lines (whether of human or non-human origin) as well as genetically engineered lymphocytes that are primary cells of non-human origin, for example and not being limited to, primary lymphocytes derived from mice, rats, monkeys, apes, cats and dogs (including NK cells and T cells such as CD3+ T cells, CD4+ T cells, CD8+ T cells, γδ T cells, invariant T cells and NK T cells).

From the more preferred primary human lymphocytes, the most preferred is a primary human T cell. Therefore, the invention also provides primary human T cell genetically engineered to express a chemokine receptor 8 polypeptide (CCR8) or a functional variant thereof, i.e., genetically engineered to express (a) a CCR8 polypeptide having the amino acid sequence of SEQ ID NO:1; (b) an amino acid variant CCR8 polypeptide having an amino acid sequence at least 85% identical to SEQ ID NO:1, which is further characterized by having CCR8 activity; or (c) a fragment of the polypeptide of (a) or (b), wherein the fragment is characterized by having CCR8 activity. The genetically engineered primary human T cell disclosed herein may be produced by a method comprising

(a) introducing into the primary human T cell
(i) an exogenous polynucleotide encoding SEQ ID NO:1;
(ii) an exogenous polynucleotide encoding a functional variant of SEQ ID NO:1, which may be
   (1) a polypeptide having an amino acid sequence at least 85% identical to SEQ ID NO:1 and which is further characterized in having CCR8 activity; or
   (2) a fragment of the polypeptide encoded by polynucleotide of (i) or (ii)(1), which fragment is characterized in having CCR8 activity;
(iii) a polynucleotide comprising or consisting of the nucleic acid sequence of SEQ ID NO:2: or
(iv) a polynucleotide comprising or consisting of a nucleic acid sequence having at least 85% sequence identity to SEQ ID NO:2 that encodes a polypeptide characterized in having CCR8 activity;
   (b) culturing the primary human T cell engineered according to (a) under conditions allowing the expression of the CCR8 polypeptide, the amino acid sequence variant CCR8 polypeptide or fragment of either; and (c) recovering the engineered primary human T cell.

The genetically engineered T cell of the invention, whether human or not and whether primary or not (although it is most preferred that the T cell is a primary human T cell), can be any T cell known in the art or described herein known or believed useful for adoptive cell therapies and/or known or believed to be of use in an in vitro or in vivo model system. Non-limiting examples of T cells encompassed by the invention include CD3+ T cells, CD4+ T cells, CD8+ T cells, γδ T cells, invariant T cells, NK T cells, and primary versions thereof, e.g., primary CD3+ T cells, primary CD4+ T cells, primary CD8+ T cells, primary γδ T cells, primary invariant T cells and primary invariant NK T cells.

The genetically engineered lymphocytes of the invention may either be a directly genetically engineered lymphocyte, i.e., a lymphocyte that has been directly subject to genetic engineering methods, or may be a lymphocyte derived from such a lymphocyte, e.g., a daughter cell or progeny of a lymphocyte that was directly genetically engineered. Thus, the genetically engineered lymphocyte of the invention may be a directly genetically engineered lymphocyte as well as any cell derived therefrom, such as a daughter cell obtained by culture of the directly engineered/modified lymphocyte.

The genetically engineered lymphocytes of the invention (preferably human lymphocytes, more preferably primary human lymphocytes, and most preferably primary human T cells) are envisioned for use in therapy and may be autologous (i.e., the donor from which the cells were derived and recipient are the same subject) or may be allogenic (i.e., the donor from which the cells were derived is different from the recipient). Where the cells are allogenic, they may be further genetically engineered or prepared such that they are not alloreactive. As understood in the art, and as used herein, not alloreactive (or, alternatively, non-alloreactive) indicates that the lymphocytes have been engineered (e.g., genetically engineered) such that they are rendered incapable of reacting to/recognizing allogenic (foreign) cells. Similarly, the genetically engineered lymphocytes of the invention can be additionally or alternatively engineered so as to prevent their own recognition by the recipient's immune system. As a non-limiting example in this respect, the lymphocytes of the invention may have disruption or deletion of the endogenous major histocompatibility complex (WIC). Such cells may have diminished or eliminated expression of the endogenous WIC, preventing or diminishing activation of the recipient's immune system against the autologous cells.

As understood in the art, such non-alloreactive cells are incapable of reacting to cells of a foreign host. Therefore, non-alloreactive cells derived from third-party donors may become universal, i.e. recipient independent. As explained above, the non-alloreactive cells may also comprise additional engineering rendering them incapable of eliciting an immune response and/or of being recognized by the recipient's immune system, preventing them from being rejected. Such cells that are non-alloreactive and/or that are incapable of eliciting an immune response or being recognized by the recipient's immune system may also be termed “off-the-shelf” lymphocytes as is known in the art. Lymphocytes can be rendered non-alloreactive and/or incapable of eliciting or being recognized by an immune system by any means known in the art or described herein. In the context of T cells, as a non-limiting example, non-alloreactive cells can have reduced or eliminated expression of the endogenous T cell receptor (TCR) when compared to an unmodified control cell. Such non-alloreactive T cells may comprise modified or deleted genes involved in self-recognition, such as but not limited to, those encoding components of the TCR including, for example, the alpha and/or beta chain. Similarly, the genetically engineered lymphocytes disclosed herein can additionally or alternatively have reduced or eliminated expression of the endogenous WIC when compared to an unmodified control cell. Such lymphocytes may comprise any modifications or gene deletions known in the art or described herein to minimize or eliminate antigen presentation, in particular, so as to avoid immunogenic surveillance and elimination in the recipient. As noted, non-alloreactive cells which optionally avoid immune surveillance are widely referenced in the art as “off the shelf” cells and the terms are used interchangeably herein. Such non-alloreactive/off the shelf lymphocytes may be obtained from repositories. The genetic modifications to reduce or eliminate alloreactivity (i.e., to render the cell non-alloreactive) and/or to reduce or eliminate self-antigen presentation (i.e., so as to prevent them from eliciting an immune response or being recognized by the recipient's immune system), as known in the art or described herein can be performed before, concurrently with, or subsequent to the genetic engineering to express CCR8 (SEQ ID NO:1) or a functional variant thereof. As a non-limiting example, off the shelf lymphocytes can be obtained from a repository and then engineered to express CCR8 or a functional variant thereof according to the methods described herein; in such a case, the modifications to render the lymphocyte non-alloreactive and/or incapable of eliciting an immune response and/or being recognized by the recipient's immune system were preformed prior to the genetic engineering to express CCR8 or a functional variant thereof.

The genetically engineered lymphocytes disclosed herein can also express a chimeric antigen receptor (CAR), an exogenous TCR, a further exogenous cytokine receptor (which sequence may or may not be modified relative to the endogenous/wild-type sequence), and/or an endogenous cytokine receptor having an amino acid sequence modified relative to the wild-type sequence (i.e a modified endogenous cytokine receptor). The genetic modification to the lymphocyte so as to (i) express the CAR, exogenous TCR, further exogenous cytokine receptor (modified or having the wild-type sequence), and/or modified endogenous cytokine receptor; (ii) reduce or eliminate alloreactivity (i.e., render it non-alloreactive as explained immediately above), and/or (iii) render it immunologically neutral (i.e., such that it does not elicit an immune response and/or cannot be recognized by the recipient's immune system) can be performed before, concurrently with, or subsequent to the genetic engineering to express CCR8 (SEQ ID NO:1) or a functional variant thereof. Additionally, the further genetic modifications disclosed herein can be combined with the genetic engineering in the context of CCR8 or a functional variant thereof. For example the methods of the invention encompass genetically engineering a lymphocyte to express a CCR8 polypeptide or a functional variant thereof, which lymphocyte may be further genetically modified according to none, one, two or all of the following: modified to express a CAR, modified to express an exogenous TCR, modified to express a further exogenous cytokine receptor (which sequence may or may not be modified relative to the endogenous/wild-type sequence), modified to express an endogenous cytokine receptor having an amino acid sequence modified relative to the wild-type sequence, modified to reduce or eliminate alloreactivity, and/or modified so that it does not elicit an immune response or cannot be recognized by the recipient's immune system. These further modifications may occur before, concurrently with or subsequent to the genetic engineering in connection with expression of CCR8 or a functional variant thereof.

In one aspect the genetically engineered lymphocyte as disclosed herein is further modified to express dominant-negative TGF-β receptor 2 (DNR) as known in the art, which may have the exemplary amino acid sequence encoded by SEQ ID NO:6.

As used herein the terms “does not elicit an immune response”, “cannot be recognized by the recipient's immune system”, “immunologically neutral” and/or analogous terms are not to be understood as absolutes. That is cells engineered for such activity (or lack of activity) may exhibit some immunologic activating/stimulating activity, but at reduced levels relative to the levels of a control cell prior to the relevant modifications, e.g., genetic engineering. The cells accordingly engineered will exhibit at least 50%, at least 60%, at least 70%, at least 80%, at least 90% or 100% inhibition of immune stimulatory activity relative to a control cell. Alternately or additionally, the cells accordingly engineered will exhibit at least 50%, at least 60%, at least 70%, at least 80%, at least 90% or 100% less immune response relative to a control cell. Inhibition of immune stimulatory activity or determination of immune response can be performed according to any method known in the art or described herein.

The invention also encompasses a genetically engineered lymphocyte obtainable by any method disclosed herein. In this respect the methods disclosed herein also encompass methods for expanding lymphocytes after the genetic engineering of the invention (and optional further genetic modifications as disclosed herein) as well as lymphocytes obtained after such expansion. The genetically engineered lymphocytes may be expanded by any suitable method known in the art or described herein. Non-limiting examples of methods of such expansion include exposure to one or more of (a) anti-CD3 antibodies; (b) anti-CD-28 antibodies; and (c) one or more cytokines. Where the lymphocyte is a T cell (e.g. a human T cell and most preferably a primary human T cell), the one or more cytokines can include interleukin-2 (IL-2) or interleukin-15 (IL-15).

The invention provides a method of immunotherapy for treating a disease comprising the use of the genetically engineered lymphocytes disclosed herein. Accordingly, provided is a genetically engineered lymphocyte (preferentially a human lymphocyte, more preferentially a primary human lymphocyte and most preferentially a primary human T cell) as described herein for use as a medicament. In particular, the genetically engineered lymphocytes disclosed herein are provided for use in a method of treating cancer characterized by the expression of the CCR8 ligand, CCL1, by the tumor or disease parenchyma. As is appreciated, CCL1 may or may not be expressed by the cancer cells (i.e., the diseased cells) themselves. A cancer also remains characterized by the expression of CCL1 where it is not expressed by the cancerous or diseased cells themselves, but where it is expressed by cells resident within the cancer/disease parenchyma and which are not cancer or disease cells. Such cells resident in the cancer/tumor/disease parenchyma that are not disease cells but that may express CCL1 include, but are not limited to, tumor resident immune cells or tumor infiltrating immune cells. The invention also provides the genetically modified lymphocytes as disclosed herein within a pharmaceutically acceptable carrier in the form of a pharmaceutical composition. The medicament and pharmaceutical compositions as disclosed herein are, in particular, of use in adoptive immune therapies. The medicament of the invention may comprise genetically engineered lymphocytes autologous to the subject to be treated, or may comprise genetically engineered lymphocytes allogenic to the subject to be treated. Where a medicament and/or pharmaceutical composition as disclosed herein comprises genetically engineered lymphocytes allogenic to the subject to be treated, such lymphocytes can be further genetically modified to be non-alloreactive and/or incapable of being recognized by the recipient's immune system as is known in the art or described herein.

### 3. BRIEF DESCRIPTION OF FIGURES

FIG. 1: Chemotactic activity of CCR8-transduced murine OT-1 T cells in response to a CCL1 gradient as determined in a migration assay. Control cells were OT-1 T cells transduced with GFP.

FIG. 2: Therapeutic effect of CCR8 transduction on ACT using tumor-antigen specific T cells in a Panc02-OVA murine cancer model. Control experiments were performed with vehicle solution and tumor-specific T cells prepared similarly but mock transduced.

FIG. 3: CCL1 expression by immune cells. (A) CCL1 expression by purified CD4+ or CD8+ T cells obtained from the spleen of a wild-type C57BL/6 mouse on activation with anti-CD3 and anti-CD28 antibodies. (B) CCL1 expression of antigen specific OT-1 cells on co-culture with an antigen-positive tumor cell line (Panc02-OVA).

FIG. 4: CCL1 expression as determined by ELISA in various tissues of Panc02-OVA tumor model mice at days 7, 14 and 21 post implantation relative to tumor free controls of the same age. LN, lymph node sampled at site distant from tumor (axillary or popliteal nodes); LN IL, ipsilateral lymph nodes with regard to the site of tumor implantation; LN CL, contralateral lymph nodes with regard to the site of tumor implantation.

FIG. 5: Therapeutic effect of CCR8 transduction on ACT using tumor-antigen specific T cells in a Panc02-OVA-CCL1 murine cancer model ((A) tumor volume and (B) survival). Control experiments were performed with tumor-specific T cells prepared similarly but mock transduced (i.e. GFP alone). (C) T-cell infiltration of tumor relative to LN (lymph node sampled at site distant from tumor (axillary or popliteal nodes)). P-values are depicted in the figure, * indicates p<0.1, and *** indicates p<0.001.

FIG. 6: Transduced T cell activation in response to stimulation by (i) the combination of anti-CD3 and anti-CD28 antibodies; or (ii) co-culture with an antigen positive tumor line (Panc02-EpCAM). Wild type T cells isolated from a C57Bl/6 mouse were transduced with mCherry (control), CCR8-GFP, CAR47 (an anti-EpCAM CAR)-mCherry, CAR47-CCR8, or CCR8-CAR47. Untransduced cells served as a further control. (A) Activation as determined by mean IFN-γ release. (B) Cytotoxic activity towards an antigen positive cell line (Panc02-EpCAM) as determined by LDH release. (C) Realtime cytotoxic activity towards an antigen positive cell line (Panc02-EpCAM) as determined in an xCELLigence assay.

FIG. 7: Effect of CCR8 transduction/expression on ACT using tumor-antigen specific T cells (expressing a tumor-specific CAR, CAR47) in a Panc02-EpCAM murine cancer model. (A) Tumor growth. (B) Survival as a function of time.

FIG. 8: Analysis of T cell populations in tumor and LN (lymph node sampled at site distant from tumor (axillary or popliteal nodes)) tissue in the Panc02-OVA in vivo murine model. (A) ratio of regulatory to CD4+ T cells; (B) percent of effector subtype in the regulatory T cells isolated in (A), i.e. percentage of eTreg cells; (C) TGF-β expression in the eTreg cells of (B).
FIG. 8D shows the expression of TGF-β by in vitro cultures of Panc02-OVA cells as determined by ELISA analysis of supernatant.
P-values are depicted in the figure, * indicates p<0.1, and *** indicates p<0.001.

FIG. 9 (A) Schematic of Dominant-Negative TGF-β receptor 2 (DNR); (B) Expression of DNR on T cells transduced according to the methods of Example 1. (C) Proliferation of DNR transduced cells in response to TGF-β (10 ng/ml during 24 hours), control cells are prepared similarly but mock transduced. P-values are depicted in the figure, *** indicates p<0.001.

FIG. 10 Therapeutic effect of transduction with dominant-negative TGF-β receptor 2 (DNR) on tumor-antigen specific T cell (OT-1 T cells) ACT in a Panc02-OVA murine cancer model ((A) tumor volume and (B) survival). Control experiments were performed with tumor-specific T cells prepared similarly but mock transduced (i.e. GFP alone). P-values are depicted in the figure, *** indicates p<0.001.

FIG. 11: Therapeutic effect of CCR8 and DNR transduction on ACT using tumor-antigen specific T cells (expressing a CAR specific for EpCAM, CAR47) in a Panc02-EpCAM murine syngeneic tumor model. Cells were transduced with vectors encoding anti-EpCAM CAR (CAR47)-mCherry, DNR-CAR47, CCR8-CAR47, or CCR8-DNR-CAR. Control experiments were performed with vehicle solution. (A) Tumor growth curves of treatment. (B) Survival.

FIG. 12: (A): Growth curves of SUIT-2-MSLN-CCL1 human tumors in NSG mice treated with a single i.v. injection of PBS, or 10⁷ CAR-transduced, DNR-CAR-transduced, CCR8-CAR-transduced or CCR8-DNR-CAR-transduced T cells (n=5 mice per group). (B): Tumor cells per bead were quantified by flow cytometry, ex vivo, after experiment termination on day 27 after tumor implantation (n=5 mice). FIG. 12 (C) CAR T cells per bead, normalized to tumor size in milligrams, quantified by flow cytometry, ex vivo, after experiment termination on day 27 after tumor implantation (n=5 mice). P-values are depicted in the figure, * indicates p<0.1, ** indicates p<0.01, and *** indicates p<0.001.

FIG. 13: (A) amino acid sequence of human CCR8, SEQ ID NO:1; (B) nucleotide sequence encoding human CCR8, SEQ ID NO:2; (C) amino acid sequence of murine CCR8, SEQ ID NO:3; (D) nucleotide sequence encoding murine CCR8, SEQ ID NO:4; (E) nucleotide sequence encoding anti-EpCAM CAR, SEQ ID NO:5; (F) nucleotide sequence encoding dominant-negative TGF-β receptor 2 (DNR), SEQ ID NO:6.

### 4. DETAILED DESCRIPTION

The role of C—C chemokine receptor 8 (CCR8) and its ligand CCL1 in the regulation of the immune response has recently been clarified. CCR8 has long been known to be expressed on certain T cells, and its interaction with CCL1 was believed to be involved with migration and with the induction and regulation of inflammatory responses (Soler et al., J. Immunol. 177(2006), 6940-6951). However, recent studies have clarified the CCR8-CCL1 interaction as playing a pivotal role in the attenuation of the immune response, as well as in the generation and maintenance of tolerance (Barsheshet et al., PNAS 114(2017), 6086-6091). With respect to cancers, CCR8 has now been recognized to be more highly expressed in the regulatory subset of T cells (Treg) and/or immune cells resident within the tumor than in conventional T cells, while CCL1 has been found to be more highly expressed by tumors as compared to adjacent normal tissues, in particular, also in infiltrating and/or resident immune cells (Piltas et al., Immunity 45(2016), 1122-1134). Moreover, activation of Tregs by CCL1 and/or tumor explants induced CCR8 expression, which further induces suppressive activities by an autocrine loop (Barsheshet et al., PNAS 114(2017), 6086-6091). Together, the role of CCR8 in the suppression of the immune system and tumor evasion has become evident. Despite these findings, the present inventors have surprisingly and unexpectedly found that lymphocytes genetically engineered to express CCR8 improve their therapeutic efficacy in adoptive therapeutic strategies. The methods disclosed herein are applicable to any type of lymphocyte capable of being used in adoptive therapy, including, but not limited to, natural killer (NK) cells and T cells. T cells of use in accordance with the methods disclosed herein include, for example, CD4+ T cells, CD8+ T cells, and γδ T cells.

Accordingly, provided is a primary lymphocyte, preferably a primary T cell, which has been genetically engineered to express CCR8 or a functional variant thereof. C—C chemokine receptor 8 (also known in the art as “CCR8”) is a known member of the 7-transmembrane segment superfamily of G-protein-coupled cell surface molecules, e.g., as disclosed in (WO 99/06561). The engineered primary lymphocytes provided herein are of use in therapeutic adoptive cell strategies. Accordingly, the primary lymphocytes disclosed herein are engineered to express human CCR8 or a functional variant thereof. However, as understood in the art, murine CCR8 and/or established T cell lines also have value in, for example, screening assays and model systems. Accordingly, also provided herein is a lymphocyte, which may be derived from an established T cell line, engineered to express murine or human CCR8 or a functional variant thereof. The amino acid sequence of the full length human and murine CCR8 polypeptides are known in the art, for example, human CCR8 is as defined in UniProt P51685 (https://www.uniprot.org/uniprot/P51685). As reported therein the amino acid sequences of human CCR8 is provided as SEQ ID NO:1; the sequence of murine CCR8 is provided as SEQ ID NO:3. Exemplary nucleic acid sequences encoding SEQ ID NO:1 and SEQ ID NO:3 are provided as SEQ ID NO:2 and SEQ ID NO:4, respectively.

As used herein, the term “genetically engineered to express CCR8” and analogous terms, refers to (1) a cell that has been recombinantly modified to express CCR8 or a functional variant of CCR8; as well as (2) the progeny of such a cell that maintains expression of such a polypeptide, e.g., obtainable by culture of the originally modified cell. Methods of genetically engineering cells to express polypeptides of interest are well known and routine in the art and include methods of introducing nucleic acids encoding the polypeptide in an appropriate form (e.g., in an expression vector) into cells via chemical or viral means. Therefore, a “genetically engineered” cell according to the invention generally encompasses the deliberate introduction of a nucleic acid molecule into the cell so that it will express the introduced sequence/molecule to produce a desired substance, e.g., human or murine CCR8, or a functional variant thereof. “Genetically engineered” encompasses any means of introducing the nucleic acid sequence or molecule into the cell described herein or known in the art suitable to allow expression of the encoded polypeptide. Thus, “genetically engineered” encompasses transduction methods (commonly understood to refer to the introduction of a foreign nucleic acid into a cell using a vector, including the use of a viral vector), and transfection methods (commonly understood to refer to the introduction of a foreign nucleic acid into a cell using non-viral means such as chemical- or electric-poration, microinjection, etc.). Thus, “genetically engineered” in more general terms also encompasses methods of transformation, i.e., the introduction of a gene, DNA, or RNA sequence into a host cell, such that the host cell will express the introduced gene or sequence to produce a desired substance, such as a polypeptide (e.g., CCR8 or a functional variant thereof) encoded by the introduced gene or sequence. The introduced gene or sequence can be referenced as a “cloned”, “foreign”, or “heterologous” gene or sequence; or a “transgene”. The introduced nucleic acid molecule/sequence can also comprise additional heterologous sequences including, for example, include heterologous promoters, start, stop, promoter, signal, secretion, or other sequences used by a cell's genetic machinery operatively linked to the coding sequences described herein, as well as further regulatory nucleic acid sequences well known in the art and/or described herein. The introduced gene or sequence can include nonfunctional sequences or sequences with no known function. According to the methods disclosed herein, a host cell that receives and expresses introduced DNA or RNA has been “genetically engineered”. As understood in the art, genetically engineered in the context of the methods and products described herein is equivalent to transformed, transduced and/or transfected; the genetically engineered cell is, for example, a transformant or a clone; and it is “transgenic”. The DNA or RNA introduced to the host cell can be derived from any source, including cells of the same genus or species as the host cell, or cells of a different genus or species.

### 4.1 Lymphocytes for Immunotherapy

The invention is in particular directed to a lymphocyte (preferably a human lymphocyte, more preferably a primary human lymphocyte, and most preferably a primary human T cell) that has been genetically engineered to express CCR8 or a functional variant thereof. The term “primary” and analogous terms in reference to a cell or cell population as used herein correspond to their commonly understood meaning in the art, i.e., referring to cells that have been obtained directly from living tissue (i.e. a biopsy such as a blood sample) or from a subject, which cells have not been passaged in culture, or have been passaged and maintained in culture but without immortalization. It is preferred that the engineered primary lymphocytes are engineered primary human lymphocytes. Primary cells have undergone very few population doublings, if any, subsequent to having been obtained from the tissue sample and/or subject, and are therefore more representative of the main functional components and characteristics of in situ tissues and cells as compared to continuous tumorigenic or artificially immortalized cell lines.

The lymphocytes according to the present invention can be any lymphocyte described herein or known in the art to be suitable for use, in particular, in an adoptive cell therapy. Accordingly, it is preferred that the lymphocyte of the invention is preferably a human lymphocyte, more preferably a primary human lymphocyte, and most preferably a primary human T cell. However, it is recognized that the methods of the invention may also be applicable for uses outside of therapies, such as in screening methods and/or in model systems, e.g., of use in in vitro assays or in vivo animal models. Therefore, the invention also encompasses genetically engineered non-human lymphocytes and/or genetically engineered lymphocytes derived from cell lines, which may be of human or non-human origin. Non-limiting examples of lymphocytes (which may be primary lymphocytes or derived from cell lines) include NK cells, inflammatory T-lymphocytes, cytotoxic T-lymphocytes, helper T-lymphocytes, CD4+T lymphocytes, CD8+T lymphocytes, γδ T lymphocytes, invariant T lymphocytes and NK T lymphocytes. It is preferred that the genetically engineered lymphocyte of the invention is a genetically engineered primary lymphocyte. Thus it is preferred that the cell of the invention is a genetically engineered primary NK cell or T cell, preferably a human cell, more preferably a primary human NK or T cell, and most preferably a primary human T cell, which may be, e.g., a CD8+ T cell, a CD4+-T cell, or γδ T cell. Accordingly, the invention relates to a genetically engineered primary lymphocyte, preferably human a NK cell or T cell such as a CD8+ T cell, CD4+ T cell, CD3+ T cell, δγ T cell, expressing a C—C receptor 8 (CCR8) polypeptide or a functional variant thereof (i.e., a variant of CCR8 that exhibits a CCR8 activity known in the art or described herein). In particular, such a lymphocyte has been genetically engineered to comprise and express a nucleic acid sequence encoding the CCR8 polypeptide or functional variant thereof.

The primary lymphocytes described herein can be isolated and/or obtained from a number of tissue sources, including but not limited to, peripheral blood mononuclear cells isolated from a blood sample, bone marrow, lymph node tissue, cord blood, thymus tissue, tissue from a site of infection, ascites, pleural effusion, spleen tissue, and/or tumors by any method known in the art or described herein. In a non-limiting example in the context of a T cell, a genetically engineered primary T cell of the present invention is that having been obtained and/or isolated from a T cell population from subject (preferably a human patient). Methods for isolating/obtaining specific populations of lymphocytes (including T cells) from patients or from donors are well known in the art and include as a first step, for example, isolation/obtaining a donor or patient sample known or expected to contain such cells, e.g., a blood or bone marrow sample. After isolating/obtaining the sample, the desired cells, e.g., NK cells or T cells, are separated from the other components in the sample. Methods for separating a specific population of desired cells from the sample are known and include, but are not limited to, e.g., leukapheresis for obtaining T cells from the peripheral blood sample from a patient or from a donor; isolating/obtaining specific populations from the sample using a FAC Sort apparatus; and selecting specific populations from fresh biopsy specimens comprising living lymphocytes by hand or by using a micromanipulator (see, e.g., Dudley, Immunother. 26(2003), 332-342; Robbins, Clin. Oncol. 29(20011), 917-924; Leisegang, J. Mol. Med. 86(2008), 573-58). The term “fresh biopsy specimens” refers to a tissue sample (e.g. a tumor tissue or blood sample) that has been or is to be removed and/or isolated from a subject by surgical or any other known means. The isolated/obtained cells are subsequently cultured and expanded according to routine methods known in the art for maintaining and/or expanding the desired primary cell and/or primary cell population. For example, in the context of T cells, culture may occur in the presence of an anti-CD3 antibody; in the presence of a combination of anti-CD3 and anti-CD28 monoclonal antibodies; and/or in the present of an anti-CD3 antibody, an anti-CD28 antibody and one or more cytokines, e.g. interleukin-2 (IL-2) and/or interleukin-15 (IL-15) (see, e.g., Dudley, Immunother. 26(2003), 332-342; Dudley, Clin. Oncol. 26(2008), 5233-5239).

As is well known in the art, it is also possible to isolate/obtain and culture/select one or more specific sub-populations of T cells, which methods are also encompassed by the invention. Such methods include but are not limited to isolation and culture of primary T cell sub-populations such as CD3+, CD28+, CD4+, CD8+, and γδ, as well as the isolation and culture of other primary lymphocyte populations such as NK T cells or invariant T cells. Such selection methods can comprise positive and/or negative selection techniques, e.g., wherein the sample is incubated with specific combinations of antibodies and/or cytokines to select for the desired sub-population. The skilled person can readily adjust the components of the selection medium and/or method and length of the selection using well known methods in the art. Longer incubation times may be used to isolate desired populations in any situation where there is or are expected to be fewer desired cells relative to other cell types, e.g., such as in isolating tumor infiltrating lymphocytes (TIL) from tumor tissue or from immunocompromised individuals. The skilled person will also recognize that multiple rounds of selection can be used in the disclosed methods.

Enrichment of the desired population is also possible by negative selection, e.g., achieved with a combination of antibodies directed to surface markers unique to the negatively selected cells. In a non-limiting example, cell sorting and/or selection via negative magnetic immunoadherence or flow cytometry that uses a cocktail of monoclonal antibodies directed to cell surface markers present on the cells negatively selected can be used. For example, to enrich for CD4+ T cells by negative selection, a monoclonal antibody cocktail typically including antibodies specific for CD14, CD20, CD11b, CD16, HLA-DR, and CD8 are used. The methods disclosed herein also encompass removing T regulatory cells, e.g., CD25+ T cells, from the population to be genetically engineered. Such methods include using an anti-CD25 antibody, or fragment thereof, or a CD25-binding ligand, such as IL-2.

The genetically engineered lymphocyte of the invention may be a genetically engineered autologous primary lymphocyte. The term “autologous” refers to any material isolated, derived and/or obtained from the same individual to whom it is later to be re-introduced, e.g. in the context of an autologous adoptive therapy, such as autologous adoptive T cell therapy (ACT) wherein the same individual is both the donor and recipient. Accordingly, in the context of the invention disclosed herein, the genetically engineered lymphocyte may be a genetically engineered autologous primary lymphocyte, including but not limited to a genetically engineered primary autologous NK cell or a primary autologous T cell, such as a primary autologous CD8+ T cell, a primary autologous CD4+ T cell, a primary autologous γδ T cell, a primary autologous invariant T cell or a primary autologous NK T cell. However the methods and materials disclosed herein (e.g., the genetically engineered lymphocyte) are not limited to autologous lymphocytes isolated and/or derived from the subject to be subsequently treated with the lymphocyte (and/or to the use of). The methods disclosed herein also encompass the use and production of genetically engineered allogeneic primary lymphocytes. As appreciated in the art, an “allogeneic lymphocyte” is a lymphocyte (e.g., a T cell) isolated from a donor of the same species as the recipient but not genetically identical to the recipient. Such allogenic cells can be used in adoptive therapies without or, preferably, with further modification, e.g., to reduce or inactivate the allogenic reactions in the intended recipient by the engineered T cell to the host (e.g., graft versus host reactions) as well as those immune reactions of the host against the engineered T cell (e.g., host versus graft reactions). Such modifications can be made by any method known in the art and/or described herein (such cells are known in the art and referenced herein as “non-alloreactive” or “off-the-shelf” T cells).

The donor and/or recipient of the lymphocytes as disclosed herein, including the subject to be treated with the allogenic or autologous genetically engineered primary lymphocytes, may be any living organism in which an immune response can be elicited (e.g., mammals). Examples of donors and/or recipients as used herein include humans, dogs, cats, mice, rats, monkeys and apes, as well as transgenic species thereof, and are preferably humans.

Accordingly, also provided herein is a method for the production of a genetically engineered lymphocyte (e.g. a human primary T cell) expressing a CCR8 or a functional variant thereof, comprising the steps of modifying (e.g. transducing) the cell to express CCR8 or functional variant thereof, culturing the modified cell under conditions allowing the expression of the CCR8 or functional variant thereof, and recovering said genetically engineered cell.

The genetically engineered lymphocytes of the invention are preferably cultured under controlled conditions, outside of their natural environment. In particular, the term “culturing” as used herein indicates that the engineered cells are maintained in vitro. The genetically engineered lymphocytes are cultured under conditions allowing the expression of the CCR8 or its functional variant. Conditions that allow the maintenance of lymphocytes and expression of a desired transgene therein are commonly known in the art and include, but are not limited to culture in the presence of agonistic anti-CD3- and anti-CD28 antibodies, as well as one or more cytokines such as interleukin 2 (IL-2), interleukin 7 (IL-7), interleukin 12 (IL-12) and/or interleukin 15 (IL-15). After expression of the CCR8 a functional fragment thereof, as described herein, the genetically engineered cell is recovered or otherwise isolated from the culture.

The lymphocytes as described herein may be activated and/or expanded as is known in the art. Thus, methods according to the invention may also include a step of activating and/or expanding a primary lymphocyte or lymphocyte population. This can be done prior to or after genetic engineering of the cells, using the methods well known in the art, e.g., as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. As appreciated in the art, such methods can encompass culturing the cells with appropriate agents such as agents that activate stimulatory receptors (e.g. agonistic antibodies) and/or target ligands of endogenous or recombinant receptors as routine in the art. Said cells can also be expanded by co-culturing with tissue or cells expressing target ligands of endogenous or recombinant receptors, including in vivo, for example in the subject's blood after administrating said cells to the subject.

### 4.2 CCR8 Polypeptide

The genetically engineered lymphocyte (e.g., a primary T cell) provided herein comprises a nucleic acid molecule encoding a C—C chemokine receptor 8 (CCR8) polypeptide or a functional variant thereof. CCR8 is a receptor expressed on the cell surface comprising seven transmembrane domains, and as such, only a part of the receptor is accessible from the intracellular space. Once engineered into in the lymphocyte(s), the encoded CCR8 polypeptide or functional variant thereof is expressed on the surface of the engineered cell and can be detected either directly, e.g., by flow cytometry or microscopy using anti-CCR8 antibodies (such as Monoclonal Rat IgG_{2B} Clone 191704, R&D Sytems (Minneapolis, Minn., USA) or Mouse IgG_{2a, κ} clone L263G8, Biolegend (San Diego, Calif., USA)) or CCR8 ligands, or indirectly, e.g., by assessing the engineered cells for CCR8 activity by any method known in the art and/or described herein.

The CCR8 polypeptide expressed by the genetically engineered lymphocyte may be the full length murine or, preferably, human, CCR8 polypeptide as known in the art, e.g., SEQ ID NO:3 or SEQ ID NO:1, respectively. Alternately, the CCR8 polypeptide expressed by the genetically engineered lymphocyte may be a variant of SEQ ID NO:1 or SEQ ID NO:3 further characterized by having CCR8 activity as defined herein, i.e., a functional variant of SEQ ID NO:1 or SEQ ID NO:3. The term “functional variants of CCR8” as used herein encompass fragments of the CCR8 polypeptide and/or amino acid sequence variants of the full-length polypeptide or fragment characterized by having CCR8 activity. Accordingly, the a functional variant of CCR8 may be a fragment of SEQ ID NO:1 or SEQ ID NO3, or may be a polypeptide having an amino acid sequence that is at least 85%, 86%, 87%, 88%, 89%, 90%, 91%, 92%, 93%, 94%, 95%, 96%, 97%, 98% or 99% identical to SEQ ID NO:1 or SEQ ID NO:3, or a fragment thereof. It is preferred that the functional variant of the invention is a functional variant of human CCR8, i.e., a functional variant of SEQ ID NO:1.

The cell of the invention can be genetically engineered with a nucleic acid sequence comprising SEQ ID NO:2 (which encodes SEQ ID NO:1) or fragment thereof, or with a nucleic acid sequence at least 85%, 86%, 87%, 88%, 89%, 90%, 91%, 92%, 93%, 94%, 95%, 96%, 97%, 98% or 99% identical to SEQ ID NO:2 or a fragment thereof, provided that the encoded protein is characterized by having CCR8 activity as defined herein or as is known in the art. Alternately, the cell of the invention can be genetically engineered with a nucleic acid sequence comprising SEQ ID NO:4 (which encodes SEQ ID NO:3) or fragment thereof, or with a nucleic acid sequence at least 85%, 86%, 87%, 88%, 89%, 90%, 91%, 92%, 93%, 94%, 95%, 96%, 97%, 98% or 99% identical to SEQ ID NO:4 or a fragment thereof, provided that the encoded protein is characterized by having CCR8 activity as defined herein or as is known in the art. It will be appreciated that due to the redundancy of the genetic code, many alternative nucleic acids encoding the CCR8 protein and/or a functional variant thereof, can be developed using routine methods and commonly practiced in the art. Such alternative nucleic acids and their use are encompassed by the invention, and the selection of the appropriate nucleic acid to use for genetically engineering a cell according to the methods disclosed herein is within common general knowledge.

The functional variants of CCR8 as disclosed herein are characterized in having CCR8 activity, e.g., imparting CCR8 activity to the genetically engineered cell. It is preferred that the characterizing CCR8 activity is chemotactic activity in response to CCL1 gradients. That is, genetically engineered cells expressing CCR8 and/or functional variants thereof as disclosed herein are preferably characterized by the ability to migrate towards a CCL1 gradient. The CCL1-induced chemotactic activity may be assessed in vitro (e.g., using migration assays) or in vivo (e.g., monitoring the movement and/or accumulation of the genetically engineered CCR8+ cells towards and in tumor tissue expressing CCL1 (which may be expressed by the tumor cells themselves and/or by lymphocytes resident within the tumor tissue (i.e. tumor infiltrating lymphocytes)). Methods to measure migration are extensively known in the literature (e.g., Valster A. et al., Methods 37(2005), 208-215; Soler et al., J. Immunol. 177(2006), 6940-6951) and include transwell-assays, confocal microscopy and flow cytometry for in vitro analysis; and flow cytometry, bioluminescence imaging, and immunohistochemistry for in vivo analysis. The migration capacity of the engineered cells can be measured by flow cytometry, ELISA, microscopy or any other suitable device or system (Justus et al., J. Vis. Exp. 88(2014), e51046, doi:10.3791/51046). A non-limiting example of a migration assay comprises the following steps: genetically engineered lymphocytes (e.g., primary human T cells such as CD8+ T cells) are labeled with a suitable fluorescent dye and seeded in serum free medium on the membrane of and/or in the upper well of a transwell insert in a 96 well plate. Recombinant CCL1 is added to the lower chamber. Migration of cells is allowed at 37° C. Thereafter, cells reaching the lower well are quantified (see also Example 1, infra, for an additional non-limiting example).

The characterizing CCR8 activity may also be CCL1-induced binding to an integrin, preferably ICAM-1. That is, genetically engineered cells expressing CCR8 and/or functional variants thereof as disclosed herein may be characterized by the ability to bind to the integrin ICAM-1 following stimulation/incubation with CCL1. The CCL1-induced binding activity may be assessed in vitro (e.g., as binding to isolated ICAM-1 or to ICAM-1 expressing cells) or in vivo by any suitable means known in the art or described herein.

A non-limiting example of an in vitro assay for detecting CCL1-induced ICAM-1 binding (i.e., CCR8 activity) may comprise the following steps: genetically engineered lymphocytes (e.g., primary human T cells such as CD8+ T cells) are incubated with CCL1 (e.g., recombinant human 1-309 (CCL1), PeproTech, Germany) for, e.g., ½ hour, and labeled with any marker allowing the subsequent detection of cells. For example, cells may be labeled with a suitable fluorescent dye such as Calcein, e.g., at 10 μg/ml. The stimulated and labeled cells are subsequently plated in PBS in flat bottom 96 well plates that have been previously coated with recombinant ICAM-1 (e.g., ICAM-1/CD54, R&D Systems Germany) and blocked with BSA. After incubation (e.g., for 1 h) and washing, the number of remaining cells is determined. In this respect it is not necessary that the determination of remaining cells is a quantified determination, i.e., a determination of an exact number of cells, rather a qualified determination is suitable for assessment of CCL1 induced binding (i.e., CCL8 activity). The genetically engineered lymphocytes exhibit no or weak binding to integrins prior to stimulation with chemokine (CCL1), thus detection of any binding or increased binding qualitatively relative to control cells (e.g., not having been stimulated/incubated with CCL1) will indicate CCR8 activity. For example, when using a fluorescent dye, bound cells may be lysed by any suitable means so as not to negatively impact the fluorescent label, such as incubation in a 10% Triton X-100 solution. The plates can be centrifuged, the supernatant collected, and fluorescence of the supernatant determined. The presence of fluorescence or the increase in fluorescence relative to control cells indicates CCR8 activity.

Without being bound by any particular theory, it is believed that CCR1 interaction with the recombinantly expressed CCR8 polypeptide or a functional variant thereof, induces a conformational change in the LFA-1 integrin normally expressed on leukocytes, allowing the binding of LFA-1 to ICAM-1. Thus, an alternate or additional non-limiting example of an in vitro assay for detecting CCR8 activity is to detect the presence of or an increase in the amount of active LFA-1 on the surface of the lymphocyte. This can also be detected (as also described above) as the presence of or an increase in the binding activity to ICAM-1. For example, genetically engineered lymphocytes (e.g., primary human T cells such as CD8+ T cells) are incubated with CCL1 (e.g., recombinant human 1-309 (CCL1), PeproTech, Germany) for, e.g., ½ hour, in the presence or absence of a (labeled) anti-LFA-1 antibody that does or does not block binding of LFA-1 to ICAM-1 (e.g., anti-LFA-1 antibody clone H155-78, Biolegend, Germany). The anti-LFA-1 antibody alone may allow the detection of or the increase in LFA-1 in active form, e.g., relative to a control cell and thus alone be a marker for CCR8 activity. Active LFA-1 can also be determined by also incubating the cells with 50 μl of (labeled) ICAM-1 (e.g., recombinant mouse ICAM 1/human Fc chimera (R&D Systems, Germany). Following a wash step, LFA-1 can be qualitatively or quantitatively detected by detecting the labeled ICAM-1 and/or the labeled anti-LFA-1 antibody. For example, the ICAM-1 and/or the anti-LFA-1 antibody can be labeled with a dye, which may be required to be subsequently developed, and the cells assessed for the presence of or the increase in (relative to a control cell) ICAM-1 binding and/or LFA-1 using any method known in the art, such as flow cytometry.

The term “at least X % identical to” in connection with the amino acid sequences/polypeptides and/or the nucleic acid sequences/nucleic acid molecules as used herein describes the number of matches (“hits”) of identical amino acid or nucleic acid residues of two or more aligned sequences as compared to the number of residues making up the overall length of the compared sequences (or the overall compared portions thereof). In other terms, using an alignment, for two or more sequences or subsequences, the percentage of residues that are the same (e.g., at least 84%, 85%, 86%, 87%, 88%, 89%, 90%, 91%, 92%, 93%, 94%, 95%, 96%, 97%, 98% or 99% identity) may be determined when the (sub)sequences are compared and aligned for maximum correspondence over a window of comparison, or over a designated region as measured using a sequence comparison algorithm as known in the art, or when manually aligned and visually inspected.

Examples of algorithms for use in determining sequence identity include, for example, those based on CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e., gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul, Nucl. Acids Res., 25(1977), 3389). The BLASTN program for nucleic acid sequences uses as default a word length (W) of 11, an expectation (E) of 10, M=5, N=4, and a comparison of both strands. For amino acid sequences, the BLASTP program uses as default a word length (W) of 3, and an expectation (E) of 10. The BLOSUM62 scoring matrix (Henikoff, Proc. Natl. Acad. Sci., 89(1989), 10915) uses alignments (B) of 50, expectation (E) of 10, M=5, N=4, and a comparison of both strands. Preferably the BLAST program is used in methods disclosed herein.

Nucleic acid sequences in accordance with the methods and genetically engineered cells disclosed herein, which may also be referenced herein as polynucleotides or nucleic acid molecules, include DNA, such as cDNA or genomic DNA, and RNA. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. Thus, the nucleic acid molecules may, for example, be nucleic acid molecules that have been synthesized according to conventional protocols of organic chemistry. The person skilled in the art is familiar with the preparation and the use of such nucleic acid molecules (see, e.g., Sambrook and Russel “Molecular Cloning, A Laboratory Manual”, Cold Spring Harbor Laboratory, N.Y. (2001)). Accordingly, further included are nucleic acid mimicking molecules known in the art such as synthetic or semi-synthetic derivatives of DNA or RNA and mixed polymers, both sense and antisense strands. They may contain additional non-natural or derivatized nucleotide bases, as will be readily appreciated by those skilled in the art. Such nucleic acid mimicking molecules or nucleic acid derivatives according to the invention include peptide nucleic acid (PNA), phosphorothioate nucleic acid, phosphoramidate nucleic acid, 2′-O-methoxyethyl ribonucleic acid, morpholino nucleic acid, hexitol nucleic acid (HNA) and locked nucleic acid (LNA), an RNA derivative in which the ribose ring is constrained by a methylene linkage between the 2′-oxygen and the 4′-carbon (see, for example, Braasch and Corey, Chemistry & Biology 8(2001), 1-7). PNA is a synthetic DNA-mimic with an amide backbone in place of the sugar-phosphate backbone of DNA or RNA, as described in, e.g., Nielsen et al., Science 254(1991), 1497; Egholm et al., Nature 365(1993), 666.

Furthermore, it is envisaged for further purposes that nucleic acid molecules may contain, for example, thioester bonds and/or nucleotide analogues. Said modifications may be useful for the stabilization of the nucleic acid molecule against endo- and/or exonucleases in the genetically engineered cell. In a non-limiting example, the nucleic acid molecules/sequences disclosed herein may be transcribed by an appropriate vector containing a chimeric gene, which allows for the transcription of said nucleic acid molecule/sequence in the genetically engineered cell. In this respect, it is also to be understood that such polynucleotide can be used for “gene targeting” or “gene therapeutic” approaches. In another embodiment said nucleic acid molecules/sequences are labeled. Methods for the detection of nucleic acids are well known in the art, e.g., by Southern and Northern blotting, PCR or primer extension. Such embodiments may be useful for screening methods for verifying successful introduction of the nucleic acid molecules/sequences described above during gene therapy approaches. Said nucleic acid molecules/sequence(s) may be a recombinantly produced chimeric nucleic acid sequence comprising any of the aforementioned nucleic acid sequences either alone or in combination.

It is understood that the term comprising, as used above and throughout this description, denotes that further sequences, components and/or steps (e.g., when describing a method) can be included in addition to the specifically recited sequences, components and/or steps. However, this term also encompasses that the claimed subject-matter consists of exactly the recited sequences, components and/or method steps.

### 4.3 Genetic Engineering

The genetically engineered lymphocyte may transiently or stably express the encoded CCR8 polypeptide or functional variant thereof. Additionally, the expression can be constitutive or constitutional, depending on the system used as is known in the art. The encoding nucleic acid may or may not be stably integrated into the engineered cell's genome. Methods for achieving stable integration of introduced nucleic acids encoding desired proteins are well known in the art, and the invention encompasses the use of such methods as well as those described herein. Preferably, the herein provided lymphocyte (preferably a human lymphocyte, more preferably a primary human lymphocyte, and most preferably a primary human T cell) has been genetically modified by introducing the nucleic acid molecule into the lymphocyte using a viral vector (e.g. a retroviral vector or a lentiviral vector).

Methods for genetically engineering cells (in particular lymphocytes such as T cells and NK cells) to express polypeptides of interest (e.g. cell surface receptors) are known in the art and can generally be divided into physical, chemical, and biological methods. The appropriate method for given cell type and intended use can readily be determined by the skilled person using common general knowledge. Such methods for genetically engineering cells by introduction of nucleic acid molecules/sequences encoding the polypeptide of interest (e.g., in an expression vector) include but are not limited to chemical- and electro-poration methods, calcium phosphate methods, cationic lipid methods, and liposome methods. The nucleic acid molecule/sequence to be transduced can be conventionally and highly efficiently transduced by using a commercially available transfection reagent and/or by any suitable method known in the art or described herein. In addition to methods of genetically engineering cells with nucleic acid molecules comprising or consisting of DNA sequences, the methods disclosed herein can also be performed with mRNA transfection. “mRNA transfection” refers to a method well known to those skilled in the art to transiently express a protein of interest, in the present case CCR8 or a functional variant thereof, in a lymphocyte, e.g., T cell. Accordingly, the methods herein may be used to genetically engineer a lymphocyte to transiently or stably (either constitutively or conditionally) express the polypeptide of interest. For example, with respect to mRNA transfection, lymphocytes may be electroporated with the mRNA coding for CCR8 or a functional variant thereof as described herein by using an electroporation system (such as e.g. Gene Pulser, Bio-Rad) and thereafter cultured by standard cell culture protocols (see, e.g., Zhao et al., Mol Ther. 13(2006), 151-159).

Physical methods for introducing a polynucleotide into a host cell include calcium phosphate precipitation, lipofection, particle bombardment, microinjection, electroporation, and the like; see, e.g., Sambrook et al., 2012, Molecular Cloning: A Laboratory Manual, volumes 1-4, Cold Spring Harbor Press, NY.

Biological methods for introducing a polynucleotide of interest into a host cell include the use of DNA and RNA vectors. Viral vectors, and especially retroviral vectors, have become the most widely used method for inserting genes into mammalian (e.g., human cells such as a T cells). Accordingly, retroviral vectors are preferred for use in the methods and cells disclosed herein. Viral vectors can be derived from a variety of different viruses, including but not limited to lentivirus, poxviruses, herpes simplex virus I, adenoviruses and adeno-associated viruses; see, e.g. U.S. Pat. Nos. 5,350,674 and 5,585,362. Non-limiting examples of suitable retroviral vectors for transducing T cells inlcude SAMEN CMV/SRa (Clay et al., J. Immunol. 163(1999), 507-513), LZRS-id3-IHRES (Heemskerk et al., J. Exp. Med. 186(1997), 1597-1602), FeLV (Neil et al., Nature 308(1984), 814-820), SAX (Kantoff et al., Proc. Natl. Acad. Sci. USA 83(1986), 6563-6567), pDOL (Desiderio, J. Exp. Med. 167(1988), 372-388), N2 (Kasid et al., Proc. Natl. Acad. Sci. USA 87(1990), 473-477), LNL6 (Tiberghien et al., Blood 84(1994), 1333-1341), pZipNEO (Chen et al., J. Immunol. 153(1994), 3630-3638), LASN (Mullen et al., Hum. Gene Ther. 7(1996), 1123-1129), pG1XsNa (Taylor et al., J. Exp. Med. 184(1996), 2031-2036), LCNX (Sun et al., Hum. Gene Ther. 8(1997), 1041-1048), SFG (Gallardo et al., Blood 90(1997), LXSN (Sun et al., Hum. Gene Ther. 8(1997), 1041-1048), SFG (Gallardo et al., Blood 90(1997), 952-957), HMB-Hb-Hu (Vieillard et al., Proc. Natl. Acad. Sci. USA 94(1997), 11595-11600), pMV7 (Cochlovius et al., Cancer Immunol. Immunother. 46(1998), 61-66), pSTITCH (Weitjens et al., Gene Ther 5(1998), 1195-1203), pLZR (Yang et al., Hum. Gene Ther. 10(1999), 123-132), pBAG (Wu et al., Hum. Gene Ther. 10(1999), 977-982), rKat.43.267bn (Gilham et al., J. Immunother. 25(2002), 139-151), pLGSN (Engels et al., Hum. Gene Ther. 14(2003), 1155-1168), pMP71 (Engels et al., Hum. Gene Ther. 14(2003), 1155-1168), pGCSAM (Morgan et al., J. Immunol. 171(2003), 3287-3295), pMSGV (Zhao et al., J. Immunol. 174(2005), 4415-4423), or pMX (de Witte et al., J. Immunol. 181(2008), 5128-5136). Most preferred are lentiviral vectors. Non-limiting examples of suitable lentiviral vectors for transducing T cells are, e.g. PL-SIN lentiviral vector (Hotta et al., Nat Methods. 6(2009), 370-376), p156RRL-sinPPT-CMV-GFP-PRE/Nhel (Campeau et al., PLoS One 4(2009), e6529), pCMVR8.74 (Addgene Catalogoue No.:22036), FUGW (Lois et al., Science 295(2002), 868-872, pLVX-EF1 (Addgene Catalogue No.: 64368), pLVE (Brunger et al., Proc Natl Acad Sci USA 111(2014), E798-806), pCDH1-MCS1-EF1 (Hu et al., Mol Cancer Res. 7(2009), 1756-1770), pSLIK (Wang et al., Nat Cell Biol. 16(2014), 345-356), pLJM1 (Solomon et al., Nat Genet. 45(2013), 1428-30), pLX302 (Kang et al., Sci Signal. 6(2013), rs13), pHR-IG (Xie et al., J Cereb Blood Flow Metab. 33(2013), 1875-85), pRRLSIN (Addgene Catalogoue No.: 62053), pLS (Miyoshi et al., J Virol. 72(1998), 8150-8157), pLL3.7 (Lazebnik et al., J Biol Chem. 283(2008), 11078-82), FRIG (Raissi et al., Mol Cell Neurosci. 57(2013), 23-32), pWPT (Ritz-Laser et al., Diabetologia. 46(2003), 810-821), pBOB (Marr et al., J Mol Neurosci. 22(2004), 5-11), and pLEX (Addgene Catalogue No.: 27976).

The invention also encompasses vectors comprising nucleic acid molecules encoding CCR8 or a functional variant thereof. As used herein, the term “vector” relates to a circular or linear nucleic acid molecule that can autonomously replicate in a host into which it has been introduced. The term “vector” as used herein particularly refers to a plasmid, a cosmid, a virus, a bacteriophage and other vectors commonly used in genetic engineering as described herein or as is known in the art. Preferably, the disclosed vectors are suitable for the transformation of lymphocytes, preferably human lymphocytes and more preferably human primary lymphocytes, including but not limited to NK cells and T cells such as CD8+ T cells, CD4+ T cells, CD3+ T cells, γδ T cells, invariant T cells and NK T cells. Vectors of use in connection with the present invention comprise a nucleic acid sequence encoding the full length CCR8 peptide or a functional variant thereof. As such, the vectors of use in connection with the present invention may encode the amino acid sequence SEQ ID NO:1, SEQ ID NO:3 or a functional variant of either, provided that the variant is characterized by CCR8 activity. In this respect, the vectors of use in connection with the present invention may comprise the amino acid sequence SEQ ID NO:2, SEQ ID NO:4, or a variant thereof, provided that the variant encodes a polypeptide characterized by CCR8 activity.

It will be appreciated that the vectors disclosed herein may contain additional sequences to allow function such as replication or expression of a desired sequence in the cell system. For example, the vectors may comprise the nucleic acid molecule encoding CCR8 or a functional variant thereof, under the control of regulatory sequences. The term “regulatory sequence” refers to DNA sequences that are necessary to effect the expression of coding sequences to which they are operably linked. As is understood in the art, the nature of such control sequences differs depending upon the host organism. In prokaryotes, control sequences generally include promoters, ribosomal binding sites, and terminators. In eukaryotes control sequences generally include promoters, terminators and, in some instances, enhancers, transactivators and/or transcription factors. The term “control sequence” is intended to include, at a minimum, all components the presence of which are necessary for expression, and may also include additional advantageous components, e.g., to allow replication. Regulatory or control sequences (including but not limited to promoters, transcriptional enhancers and/or sequences), which allow for induced or constitutive expression of the CCR8, or its variant or fragment as described herein, may be employed. Suitable promoters include but are not limited to the CMV promoter, the UBC promoter, PGK, the EF1A promoter, the CAGG promoter, the SV40 promoter, the COPIA promoter, the ACTSC promoter, or the TRE promoter (e.g., as disclosed in Qin et al., PLoS One. 5(2010), e10611); the Oct3/4 promoter (e.g., as disclosed in Chang et al., Molecular Therapy 9(2004), S367-S367 (doi: 10.1016/j.ymthe.2004.06.904)); or the Nanog promoter (e.g., as disclosed in Wu et al., Cell Res. 15(2005), 317-24).

The vectors of use in the present invention are preferably expression vectors. Suitable expression vectors have been widely described in the literature and the determination of the appropriate expression vector can be readily made by the skilled person using routine methods. Preferably, the vectors disclosed herein comprises a recombinant polynucleotide (i.e., a nucleic acid sequence encoding the CCR8 or a functional variant) as well as expression control sequences operably linked to the nucleotide sequence to be expressed. The vectors as provided herein preferably further comprise a promoter. The herein described vectors may also comprise a selection marker gene and a replication-origin ensuring replication in the host (i.e. a genetically engineered (e.g., transduced) lymphocyte such as a T cell). Moreover, the herein provided vectors may also comprise a termination signal for transcription. Between the promoter and the termination signal may be at least one restriction site or a polylinker to enable the insertion of a nucleic acid molecule encoding a polypeptide desired to be expressed (e.g. a nucleic acid sequence encoding the CCR8 or a functional variant thereof). The use of expression vectors, including insertion of the encoding nucleic acid molecule/sequence and the harvest of the expressed polypeptide, is routine in the art. Non-limiting examples of vectors suitable for use in the present invention include cosmids, plasmids (e.g., naked or contained in liposomes) and viruses (e.g., lentiviruses, retroviruses, adenoviruses, and adeno-associated viruses) that incorporate the nucleic acid molecules encoding CCR8, or a functional variant or fragment thereof. Of preferred use is a viral vector.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). Alternately, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids may be naturally occurring or synthetic lipids. Lipids suitable for use in methods of nucleic acid molecule delivery to a host cell (i.e., to genetically engineer the host cell) can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.).

Regardless of the method used to introduce exogenous nucleic acids into a host cell, in order to confirm the presence of the recombinant DNA sequence in the target cell (i.e., to confirm that the cell has been genetically engineered according to the methods disclosed herein), a variety of assays may be performed. Such assays include, for example, “molecular biological” assays well known to those of skill in the art, such as Southern and Northern blotting, RT-PCR and PCR; “biochemical” assays, such as detecting the presence or absence of a particular polypeptide, e.g., by immunological means (ELISAs and/or Western blots) or by assays described herein to identify whether the cell exhibits a property or activity associated with the engineered polypeptide, i.e. assays to assess whether the lymphocyte (more preferably a human primary lymphocyte such as an NK cell or T cell) exhibits CCR8 activity.

The genetic engineering methods disclosed herein are applied to lymphocytes, preferably T cells. As known in the art, T cells are cells of the adaptive immune system that recognize their target in an antigen specific manner. These cells are characterized by surface expression of CD3 and a T cell receptor (TCR), which recognizes a cognate antigen in the context of a major histocompatibility complex (MHC). T cells may be further subdivided in CD4+ or CD8+ T cells. CD4+ T cells recognize an antigen through their TCR in the context of MHC class II molecules that are predominantly expressed by antigen-presenting cells. CD8+ T cells recognize their antigen in the context of MHC class I molecules that are present on most cells of the human body. Methods for identifying, separating and maintaining specific subpopulations of T cells (e.g., as a culture of primary T cells) such as CD3+, CD4+ and/or CD8+ T cells from a cell population (such as a population of peripheral blood mononuclear cells e.g., having been isolated from a patient for the purpose of autologous cell therapy) are well known to those skilled in the art and include flow cytometry, microscopy, immunohistochemistry, RT-PCR or western blot (Kobold, J Natl Cancer Inst 107(2015), 107).

As described herein, the genetically engineered lymphocyte of the present invention is recombinantly modified with a nucleic acid sequence encoding (and driving/permitting expression of) the herein described CCR8 or a functional variant thereof. In the case of cells bearing natural anti-tumor specificity (such as tumor-infiltrating lymphocytes (TIL see, e.g., Dudley et al., J Clin Oncol. 31(2013), 2152-2159)) or antigen-specific cells sorted from the peripheral blood of patients for their tumor-specificity by flow cytometry (Hunsucker et al., Cancer Immunol Res. 3(2015), 228-235), the genetically engineered cells described herein may only be modified to express CCR8 or the functional variant thereof. However, the genetically engineered T cell of the invention may be further engineered with additional nucleic acid molecules to express, in addition to the exogenous CCR8 or functional variant thereof, other polypeptides of use in ACT, e.g., with a nucleic acid sequence encoding a further, exogenous, T cell receptor, a chimeric antigen receptor (CAR) specific for a tumor of interest, an exogenous cytokine receptor (which sequence may or may not be modified relative to the endogenous/wild-type sequence), and/or an endogenous cytokine receptor having a sequence modified relative to the wild-type sequence (i.e a modified endogenous cytokine receptor). Alternately or additionally, the T cell of the invention can be further genetically modified to disrupt the expression of the endogenous T cell receptor, such that it is not expressed or expressed at a reduced level as compared to a T cell absent such modification.

As used herein, an “exogenous T cell receptor” or “exogenous TCR” refers to a TCR whose sequence is introduced into the genome of a lymphocyte (e.g., a human primary T cell) that may or may not endogenously express the TCR. Expression of an exogenous TCR on an immune effector cell can confer specificity for a specific epitope or antigen (e.g., an epitope or antigen preferentially present on the surface of a cancer cell or other disease-causing cell). Such exogenous T cell receptors can comprise alpha and beta chains or, alternatively, may comprise gamma and delta chains. Exogenous TCRs useful in the invention may have specificity to any antigen or epitope of interest. Examples of such exogenous TCRs include, but are not limited to, receptors recognizing WT1 (Wilms tumor specific antigen 1; see, e.g., Sugiyama, Japanese Journal of Clinical Oncology 40(2010), 377-87); receptors recognizing MAGE (see, e.g., WO 2007/032255), receptors recognizing SSX (see, e.g., Y Zhou et al., J. Natl. Cancer Inst. 97(2005), 823-835), receptors recognizing NY-ESO-1 (see, e.g., WO 2005/113595) and receptors recognizing HER2neu (see, e.g., WO 2011/0280894).

As used herein, the term “reduced expression” and analogous terms refer to any reduction in the expression of the endogenous T cell receptor at the cell surface of a genetically-modified cell when compared to a control cell. The term reduced can also refer to a reduction in the percentage of cells in a population of cells that express an endogenous polypeptide (i.e., an endogenous T cell receptor) at the cell surface when compared to a population of control cells. Accordingly, the term “reduced expression” in connection with the expression of an endogenous T cell receptor encompasses both a partial knockdown and a complete knockdown of the endogenous T cell receptor within the population of genetically modified cells.

The genetically modified lymphocyte of the invention, i.e., expressing CCR8 or a functional variant thereof, may be further modified to express a chimeric antigen receptor as known in the art (also referenced as a “CAR”). Chimeric antigen receptors (CARs) are well known in the art and refer to an engineered receptor that confers or grafts specificity for an antigen onto a lymphocyte (e.g., most preferably a human primary T cell). A CAR typically comprises an extracellular ligand-binding domain or moiety and an intracellular domain that comprises one or more stimulatory domains that transduce the signals necessary for lymphocyte (e.g., T cell) activation. In some embodiments, the extracellular ligand-binding domain or moiety can be in the form of single-chain variable fragments derived from a monoclonal antibody (scFvs), which provide specificity for a particular epitope or antigen (e.g., an epitope or antigen associated with cancer, such as preferentially express on the surface of a cancer cell or other disease-causing cell). The extracellular ligand-binding domain can be specific for any antigen or epitope of interest. The intracellular stimulatory domain typically comprises the intracellular domain signaling domains of non-TCR T cell stimulatory/agonistic receptors. Such cytoplasmic signaling domains can include, for example, but not limited to, the intracellular signaling domain of CD3, CD28, 4-1BB, OX40, or a combination thereof. A chimeric antigen receptor can further include additional structural elements, including a transmembrane domain that is attached to the extracellular ligand-binding domain via a hinge or spacer sequence.

As with the optionally engineered exogenous TCR, the optional CAR is to provide tumor specificity and allow for the recognition target tumor or disease cells. Suitable CARs are well known in the art, and include, but are not limited to, anti-EGFRv3-CAR (see, e.g., WO 2012/138475), anti-CD22-CAR (see, e.g., WO 2013/059593), anti-BCMA-CAR (see, e.g., WO 2013/154760), anti-CD19-CAR (see, e.g., WO 2012/079000), anti-CD123-CAR (see, e.g., US 2014/0271582), anti-CD30-CAR (see, e.g., WO 2015/028444) and anti-Mesothelin-CAR (see, e.g., WO 2013/142034).

The genetically modified lymphocyte of the invention, i.e., expressing CCR8 or a functional variant thereof, may be further modified to express one or more further exogenous cytokine receptors (which may have a wild-type sequence or may have an amino acid sequence modified relative to that of the endogenous/wild type sequence) and/or one or more endogenous cytokine receptors having a sequence modified from that of the endogenous sequence. As used herein, an “exogenous cytokine receptor” refers to a cytokine receptor whose sequence is introduced into the genome of a lymphocyte (e.g., a human primary T cell) that does not endogenously express the receptor. Similarly, “endogenous cytokine receptor” refers to a receptor whose sequence is introduced into the genome of a lymphocyte (e.g., a human primary T cell) that endogenously expresses the receptor. The introduced exogenous or endogenous cytokine receptor may be modified to alter the function of the receptor normally exhibited in its endogenous environment. For example, dominant-negative mutations to receptors are known that bind ligand but which ligand-receptor interaction does not elicit the endogenous activity normally associated with such interaction. Expression of an exogenous cytokine receptors (modified or not) and/or a modified endogenous receptors can confer ligand-specific activity not normally exhibited by the lymphocyte or, in the case of dominant-negative modifications, can act a ligand-sinks to bind cytokines and prevent and/or decrease the ligand-specific activity. One such dominant-negative receptor known in this respect is the dominant-negative TGF-β receptor 2 (DNR), a modified TGF-β receptor 2 lacking the intracellular domain of the endogenous molecule which prevents the signal transduction into the cell on TGF-β binding; see, Siegel et al., PNAS 100(2003), 8430-8435. An exemplary sequence of DNR is the amino acid sequence encoded by SEQ ID NO:6. Of particular interest is the use of DNR in the context of the invention.

### 4.4 Non-Alloreactive T Cells

The genetically engineered lymphocytes obtainable by the methods described herein (preferably a human lymphocyte, more preferably a primary human lymphocyte, and most preferably a primary human T cell) are of use as a medicament, e.g., in the treatment of cancer. The genetically engineered lymphocytes of the invention and the treatment based on their use may be either part of an autologous immunotherapy or part of an allogenic immunotherapy treatment. As understood in the art, “autologous” in the context of the genetically engineered lymphocytes and immunotherapy methods of the invention refers to the situation where the origin of the lymphocyte cell line or population used in the treatment originate from the patient to be treated, i.e., the donor of the lymphocytes and the recipient of the immunotherapy (i.e., cell transfer) are the same. “Allogenic” in the context of the genetically engineered lymphocytes and immunotherapy methods of the invention refers to the situation where the origin the lymphocytes or population of lymphocytes used for the immunotherapy originate from a genetically distinct donor as the patient.

Although the genetic engineering methods disclosed herein may be practiced with lymphocyte cell lines, e.g., T cell lines, they are preferentially intended to be practiced ex-vivo on cultured lymphocytes obtained from patients or donors, e.g., primary lymphocytes. In the case of allogenic immunotherapies, i.e., where the donor and recipient of the genetically engineered lymphocytes of the invention are not the same (not genetically identical), it is preferred that the lymphocytes are engineered to render them non-alloreactive. This is an effort to promote not only proper engraftment, but also to minimize undesired graft-versus-host immune reactions. In the context of the invention, such non-alloreactive engineering can be actively performed in combination with the other methods of genetic engineering herein, e.g., occurring before, concurrently with or subsequent to the methods of genetic engineering for expression of CCR8 or a functional variant thereof. Accordingly, the method of the invention may include steps of procuring the T-cells from a donor and inactivating genes thereof involved in WIC recognition as well known in the art. Such methods are generally reliant on disruption of the endogenous TCR. The TCR comprises two peptide chains, alpha and beta, which assemble to form a heterodimer that further associates with the CD3-transducing subunits to form the T-cell receptor complex present on the cell surface. Each alpha and beta chain of the TCR consists of an immunoglobulin-like N-terminal variable (V) and constant (C) region, a hydrophobic transmembrane domain, and a short cytoplasmic region. As for immunoglobulin molecules, the variable region of the alpha and beta chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft-versus-host immune reactions, which, when severe can present as graft-versus-host disease (GVHD). It is known that normal surface expression of the TCR depends on the coordinated synthesis and assembly of all seven components of the complex. The inactivation of TCRalpha or TCRbeta gene (and, thus, the expressed peptide) can result in the elimination of the TCR from the surface of T cells, preventing recognition of alloantigen (and, thus, GVHD) rendering the cells non-allogenic.

Alternatively the non-alloreactive engineering methods can have been performed separately, such as to establish a universal, patient-independent source or cells, e.g., as would be available for purchase from a depository of prepared cells. Accordingly, the invention also encompasses the use of lymphocytes (i.e., off the shelf lymphocytes), preferably primary lymphocytes, purchased from depositories and/or that have already been engineered for non-alloreactivity prior to the genetic engineering methods disclosed herein, i.e., engineering to express CCR8 or a functional variant thereof, and optional engineering to express an exogenous TCR or CAR. Accordingly, the methods disclosed herein are applicable to primary lymphocytes, in particular primary human T cells or NK cell, that are non-allogenic, i.e., “off-the-shelf” primary human lymphocytes such as T cells or NK cells.

In a similar manner the genetically engineered cells of the invention can be additionally or alternatively further engineered to eliminate or reduce the ability to elicit an immune response, and/or to eliminate or reduce recognition by the host immune system. This is an effort to minimize or eliminate host-versus-graft immune reactions. As with the non-alloreactive engineering, the engineering of the cells to reduce or eliminate the susceptibility to the host immune system (and/or the ability to elicit a host immune reaction) can be performed before, concurrently with, or after any other engineering methods as disclosed herein. As a non-limiting exemplary embodiment, engineering the cells to reduce or eliminate the susceptibility to the host immune system (and/or the ability to elicit a host immune reaction) can be performed by reducing or eliminating expression of the endogenous major histocompatibility complex.

### 4.5 Therapeutic Applications

The genetically engineered lymphocytes (preferably a human lymphocyte, more preferably a primary human lymphocyte, and most preferably a primary human T cell), obtainable by the methods disclosed herein are envisioned as for use as a medicament in the treatment of diseases including, but not limited to, cancers or precancerous conditions characterized by the expression of the CCR8 ligand CCL1. “Characterized by the expression of CCL1” as used herein indicates that the cancerous or precancerous parenchyma taken as a whole expresses CCL. Accordingly, a cancer or precancerous tissue is characterized by the expression of CCL1 not only where the cancerous or precancerous cells themselves express CCL1, but also wherein any cells within the diseased parenchyma express CCL1. For example, a cancer or pre-cancer is also characterized by the expression of CCL1 where the cancer or precancerous cells do not express CCL1, but where immune cells resident within the diseased tissue express CCL1 (e.g., infiltrating lymphocytes, in particular tumor infiltrating lymphocytes (TIL)). The term “cancer” or “proliferative disease” as used herein means any disease, condition, trait, genotype or phenotype characterized by unregulated cell growth or replication as is known in the art. Because the characteristic feature of the cancer/proliferative disease or precancerous condition according to the methods and uses disclosed herein (i.e., characterized by expression of CCL1) is not necessarily dependent on the expression profile of the cancer or pre-cancer cells per se (i.e., the characterizing expression of CCL1 can be satisfied by tumor resident cells, in particular, immune cells) the cancers/proliferative diseases that can be treated according to the methods and with the genetically engineered lymphocytes disclosed herein include all types of tumors, lymphomas, and carcinomas provided that they exhibit tumor parenchyma and/or tumor cells expressing CCL1. Accordingly, provided is a method for treating a disease wherein the cancer, pre-cancer, or proliferative disease cells (i) are negative for CCL1 expression, (ii) are positive for CCL1 expression; or (iii) partially positive for CCL1 expression (i.e., only some of the diseased cells express CCL1), provided that where the diseased cells do not express CCL1, some other cell within the tumor parenchyma expresses CCL1, e.g., TILs.

Non-limiting examples of such cancers include colorectal cancer, brain cancer, ovarian cancer, prostate cancer, pancreatic cancer, breast cancer, renal cancer, nasopharyngeal carcinoma, hepatocellular carcinoma, melanoma, skin cancer, oral cancer, head and neck cancer, esophageal cancer, gastric cancer, cervical cancer, bladder cancer, lymphoma, chronic or acute leukemia (such as B, T, and myeloid derived), sarcoma, lung cancer and multidrug resistant cancer.

The terms “treatment”, “treating” and the like are used herein to generally mean obtaining a desired pharmacological and/or physiological effect. The effect may be prophylactic in terms of completely or partially preventing a disease or symptom thereof, and/or may be therapeutic in terms of partially or completely curing the disease or condition, and/or adverse effect attributed to the disease or condition. The term “treatment” as used herein covers any treatment of a disease or condition in a subject and includes: (a) preventing and/or ameliorating a proliferative disease (preferably cancer) from occurring in a subject that may be predisposed to the disease; (b) inhibiting the disease, i.e., arresting its development, such as inhibition of cancer progression; (c) relieving the disease, i.e. causing regression of the disease, such as the repression of cancer; and/or (d) preventing, inhibiting or relieving any symptom or adverse effect associated with the disease or condition. Preferably, the term “treatment” as used herein relates to medical intervention of an already manifested disorder, e.g., the treatment of a diagnosed cancer.

The treatment or therapy (i.e., comprising the use of a medicament/pharmaceutical composition comprising a genetically engineered lymphocyte as disclosed herein) may be administered alone or in combination with appropriate treatment protocols for the particular disease or condition as known in the art. Non-limiting examples of such protocols include but are not limited to, administration of pain medications, administration of chemotherapeutics, therapeutic radiation, and surgical handling of the disease, condition or symptom thereof. Accordingly the treatment regimens disclosed herein encompass the administration of the genetically engineered lymphocyte expressing a CCR8 or functional variant thereof together with none, one, or more than one treatment protocol suitable for the treatment or prevention of a disease, condition or a symptom thereof, either as described herein or as known in the art. Administration “in combination” or the use “together” with other known therapies encompasses the administration of the medicament/pharmaceutical composition comprising a genetically engineered lymphocyte as disclosed herein before, during, after or concurrently with any of the co-therapies disclosed herein or known in the art. The genetically engineered lymphocytes disclosed herein (or the pharmaceutical composition/medicament comprising such lymphocytes) can be administered alone or in combination with other therapies or treatments during periods of active disease, or during a period of remission or less active disease.

When administered in combination, the genetically engineered lymphocyte immunotherapy (e.g., ACT) and/or any additional therapy, can be administered in an amount or dose that is higher, lower or the same than the amount or dosage where each therapy or agent would be used individually, e.g., as a monotherapy. In certain embodiments, the administered amount or dosage of the genetically engineered lymphocyte therapy, and/or at least one additional agent or therapy is lower (e.g., at least 20%, at least 30%, at least 40%, or at least 50%) than the amount or dosage of the corresponding therapy(ies) or agent(s) used individually.

The methods described herein employ a medicament comprising a genetically engineered lymphocyte (preferably a human lymphocyte and more preferably a primary human lymphocyte such as a T cell or NK cell) that has been recombinantly modified ex vivo to express CCR8 or a functional variant thereof. In the disclosed methods, the genetically engineered lymphocyte is adoptively transferred into the subject. Alternately or additionally, the genetically engineered lymphocyte is pulsed with tumor antigen prior to modification with the nucleic acid molecule.

To generate cells for adoptive transfer, the above-described nucleic acid molecules encoding the CCR8 or a functional variant thereof and optionally a second construct for co-expression of a tumor specific TCR or CAR and/or a third construct for inactivating the endogenous TCR are delivered to lymphocytes, in particular, T cells, according to suitable methods to allow expression of the CCR8 or variant, and, optionally, the expression of the tumor specific TCR or CAR and/or inactivation of the endogenous TCR. The engineered lymphocytes are “anti-tumor lymphocytes” (e.g., “anti-tumor T cells), which are able to become activated and expand in response to a tumor antigen. Anti-tumor T cells, useful for adoptive T cell transfer include, but are not limited to peripheral blood derived T cells expressing endogenous receptors that recognize and respond to tumor antigens, or that have been genetically modified to express such receptors, e.g. CARs. As will be appreciated, and as has been detailed herein, the genetically engineered lymphocytes may be autologous or allogenic. Autologous lymphocytes for use in the methods disclosed herein also include immune cells obtained from resected tumors. The lymphocyte may be a polyclonal or monoclonal tumor-reactive T cell, i.e., obtained by apheraesis, and may be expanded ex vivo against tumor antigens presented by autologous or artificial antigen-presenting cells.

The methods provided herein involve adoptive cell therapy and comprise administering a genetically engineered lymphocyte as described in detail herein that has or is expected to have anti-tumor activity. Prior to administration, the genetically engineered lymphocytes may be expanded as known in the art. Exemplary methods of such expansion include culture in the presence of (stimulating) cytokines such as interleukin-2 (IL-2) and/or interleukin-15 (IL-15). Such expansion may also be performed in the presence of interleukin-12 (IL-12), interleukin-7 (IL-7), interleukin-21 (IL-21), anti-CD3 antibodies, and/or anti-CD28 antibodies.

The genetically engineered lymphocytes may further be rendered resistant to chemotherapy drugs that are used as standards of care as described herein or known in the art. Engineering such resistance into the lymphocytes of the invention is expected to help the selection and expansion of the engineered lymphocytes in-vivo undergoing chemotherapy or immunosuppression.

The genetically engineered lymphocytes of the invention may undergo robust in vivo T cell expansion upon administration to a patient, and may remain persist in the body fluids for an extended amount of time, preferably for a week, more preferably for 2 weeks, even more preferably for at least one month. Although the genetically engineered lymphocytes according to the invention are expected to persist during these periods, their functional life span is not expected to exceed more than a year, no more than 6 months, no more than 2 months, or no more than one month. The cells of the invention may also be additionally engineered with safety switches that allow for potential control of the cell therapeutics. Such safety switches of potential use in cell therapies are known in the art and include (but are not limited to) the engineering of the cells to express targets allowing antibody depletion (e.g., truncated EGFR; Paszkiewicz et al., J Clin Invest 126(2016), 4262-4272), introduction of artificial targets for small molecule inhibitors (e.g., HSV-TK; Liang et al., Nature 563(2018), 701-704) and introduction of inducible cell death genes (e.g., icaspase; Minagawa et al., Methods Mol Biol 1895(2019), 57-73).

The administration of the lymphocytes or population of lymphocytes according to the present invention may be carried out in any convenient manner, including by aerosol inhalation, injection, ingestion, transfusion, implantation or transplantation. The medicaments and compositions described herein may be administered subcutaneously, intradermaly, intratumorally, intranodally, intramedullary, intramuscularly, by intravenous or intralymphatic injection, or intraperitoneally. The lymphocytes, medicament and/or compositions of the present invention are preferably administered by intravenous injection.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. For example, the genetically engineered lymphocytes of the invention may be administered to the subject at a dose of 10⁴ to 10¹⁰ T cells/kg body weight, preferably 10⁵ to 10⁶ T cells/kg body weight. In the context of the present invention the lymphocytes may be administered in such a way that an upscaling of the T cells to be administered is performed by starting with a subject dose of about 10⁵ to 10⁶ T cells/kg body weight and then increasing to dose of 10¹⁰ T cells/kg body weight. The cells or population of cells can be administrated in one or more doses.

### 4.6 Pharmaceutical Compositions

The term “medicament” is used interchangeably with the term “pharmaceutical composition” and relates to a composition suitable for administration to a patient, preferably a human patient. Accordingly, the invention provides genetically engineered lymphocytes, such as NK cells and T cells including CD3+ T cells, CD8+ T cells, CD4+ T cells, γδ T cells, invariant T cells and NK T cells, expressing a CCR8 or a functional variant thereof, or such engineered lymphocytes produced/obtainable by the method disclosed herein for use as a medicament. The medicament/pharmaceutical composition may be administered to an allogenic recipient, i.e. to recipient that is a different individual from that donating the T cells, or to an autologous recipient, i.e. wherein the recipient patient also donated the T cells. Alternately the medicament/pharmaceutical composition may comprise non-allogenic lymphocytes, (“off the shelf” lymphocytes as known in the art). Regardless of the species of the patient, the donor and recipient (patient) are of the same species. It is preferred that the patient/recipient is a human.

In the manufacture of a pharmaceutical formulation according to the invention, the genetically engineered lymphocytes are typically admixed with a pharmaceutically acceptable carrier excipient and/or diluent and the resulting composition is administered to a subject. The carrier must, of course, be acceptable in the sense of being compatible with any other ingredients in the formulation and must not be deleterious to the subject or engineered cells. Examples of suitable pharmaceutical carriers are well known in the art and include phosphate buffered saline solutions, water, emulsions, such as oil/water emulsions, various types of wetting agents, sterile solutions etc. The carrier may be a solution that is isotonic with the blood of the recipient. Compositions comprising such carriers can be formulated by well known conventional methods. The pharmaceutical compositions of the invention can further comprise one or more additional agents useful in the treatment of a disease in the subject. Where the genetically-modified lymphocyte is a primary human T cell (or a cell derived therefrom), pharmaceutical compositions of the invention can further include biological molecules, such as cytokines (e.g., IL-2, IL-7, IL-15, and/or IL-21), which promote in vivo cell proliferation and engraftment. The genetically modified lymphocytes of the invention can be administered in the same composition as the one or more additional agent or biological molecule or, alternatively, can be co-administered in separate compositions.

Also provided herein is a kit comprising a nucleic acid molecule, a vector and/or a genetically modified lymphocyte of the invention as described herein. Accordingly, the kit may comprise one or more of (i) a nucleic acid encoding a CCR8 polypeptide, e.g. having the amino acid sequence of SEQ ID NO:1 or SEQ ID NO:3 (preferably human CCR8, SEQ ID NO:1); (ii) a nucleic acid encoding a fragment of a CCR8 polypeptide, e.g. having the amino acid sequence of a fragment of SEQ ID NO:1 or SEQ ID NO:3 (preferably human CCR8, SEQ ID NO:1), characterized in having CCR8 activity; (iii) encoding a CCR8 polypeptide or fragment thereof having an amino acid sequence at least 85% identical to the encoded sequence of (i) or (ii) characterized in having CCR8 activity; (iv) comprising or consisting of the nucleic acid sequence of SEQ ID NO:2 or SEQ ID NO:4 (preferably human CCR8, SEQ ID NO:2); (v) comprising or consisting of a fragment of the nucleic acid sequence of SEQ ID NO:2 or SEQ ID NO:4 (preferably human CCR8, SEQ ID NO:2), which encodes a polypeptide characterized in having CCR8 activity; (vi) comprising or consisting of a nucleic acid sequence having at least 85% identity to the nucleic acid sequence of (iv) or (v), which encodes a polypeptide characterized in having CCR8 activity; (vii) a vector comprising a nucleic acid molecule according to (i) to (vi), above; or a genetically modified lymphocyte, e.g., a primary human lymphocyte such as a T cell comprising the nucleic acid molecule according to (i) to (vi), above, or expressing a polypeptide encoded by a nucleic acid molecule according to (i) to (vi), above. The herein provided treatment methods may be realized by using the kit. Thus also provided is a kit as described above for use in the treatment of a disease or condition characterized by the expression of CCL1. Advantageously, the herein described kit further comprises optionally (a) reaction buffer(s), storage solutions (i.e., preservatives), wash solutions and/or remaining reagents or materials required for the performance of the methods disclosed herein. Parts of the kit of the invention can be packaged individually in vials or bottles or in combination in containers or multicontainer units. In addition, the kit may contain instructions for use. The manufacture of the described kit preferably follows standard procedures, which are known to the person skilled in the art.

The pharmaceutical compositions described herein can be used in combination with a chemotherapeutic agent. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General chemotherapeutic agents considered for use in combination therapies include anastrozole, bicalutamide, bleomycin sulfate, busulfan, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, Gemcitabine, hydroxyurea (Hydrea®), Idarubicin, ifosfamide, irinotecan, L-asparaginase, leucovorin calcium, melphalan, 6-mercaptopurine, methotrexate, mitoxantrone, mylotarg, paclitaxel, Yttrium90/MX-DTPA, pentostatin, tamoxifen citrate, teniposide, 6-thioguanine, thiotepa, tirapazamine, topotecan hydrochloride, vinblastine, vincristine, and vinorelbine.

Anti-cancer agents of particular interest for combination with the genetically engineered lymphocyte based methods and compounds disclosed herein include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatin, pemetrexed, raltitrexed, cladribine, clofarabine, azacitidine, decitabine and gemcitabine.

Exemplary alkylating agents include, without limitation, nitrogen mustards, uracil mustard, ethylenimine derivatives, alkyl sulfonates, nitrosoureas, triazenes, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, dacarbazine, oxaliplatin, temozolomide, dactinomycin, melphalan, altretamine, carmustine, bendamustine, busulfan, carboplatin, lomustine, cisplatin, chlorambucil, cyclophosphamide, dacarbazine, altretamine, ifosfamide, prednumustine, procarbazine, mechlorethamine, streptozocin, thiotepa, cyclophosphamide, and bendamustine HCl.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

### 5. EXAMPLES

### 5.1 Example 1: Activity and Functionality of CCR8 Transduced T Cells

The following example demonstrates the activity and functionality imparted to T cells engineered to express CCR8.

### Animals

C57BL/6 mice were purchased from Charles River. Mice transgenic for a T cell receptor specific for ovalbumin (OT-1) were obtained from the Jackson laboratory, USA (Stock number 003831) and bred at the animal facilities at the Klinikum der Universitat Munchen. Animals were housed in specific pathogen-free facilities and all experimental studies were approved and performed in accordance with guidelines and regulations implemented by the Regierung von Oberbayern. For survival studies the age of mice at euthanasia mandated by a moribund state of health was recorded in Kaplan Meyer plots.

### T Cell Isolation and Culture

Wild-type (from C57BL/6 mice) or OT-1 T cells (specific for OVA antigen) were isolated and processed as follows. Primary splenocytes were harvested and processed to single cell suspensions by passing through 100 μm strainers and treatment with erythrocyte lysis buffer. Cells were then counted and cultured for 24 hours with RPMI medium supplemented with 10% FCS, 100 U/ml Pen/Strep, 2 mM L-Glutamine, 1 mM HEPES, 1 mM Sodium Pyruvate and 50 μM β-mercaptoethanol (all reagents from Gibco) (T cell medium), further supplemented with anti-CD3 and anti-CD28 antibodies at 1 μg/ml each (eBioscience, now ThermoScientific, clones 145-2C11 and 37.51, respectively).

T cells were subsequently transduced as follows. The retroviral vector pMP71 (Schambach et al., Mol Ther 2(2000), 435-45; EP-B1 0 955 374) was used for transfection of the ecotrophic packaging cell line Plat-E. For migration experiments, T cells were transduced with a CCR8-GFP fusion polypeptide (CCR8-2A-GFP; SEQ ID NO:3) to allow detection of expression by flow-cytometry. For other in vitro or in vivo studies, unless otherwise indicated, T cells were transduced with the nucleotide sequence SEQ ID NO:2, encoding the CCR8 polypeptide (SEQ ID NO:1).

Transduction was performed according to the method described by Leisegang et al. J Mol Med 86(2008), 573; Mueller et al. J Virol 86(2012), 10866-10869; and Kobold et al., J Natl Cancer Inst 107(2015), 364. Briefly, the packaging cell line Plat E (as described by Morita et al. Gene Ther 7(2000), 1063) was seeded in 6-well plates and grown over night to 70-80% confluence. On day one, 16 μg of DNA (e.g., comprising cDNA encoding CCR8, SEQ ID NO:2) was mixed with 100 mM CaCl2 (Merck, Germany) and 126.7 μM Chloroquin (Sigma, USA). Plat-E cells were starved for 30 min in low serum medium (3%) and then incubated for 6 h with the precipitated DNA. Medium was then removed and exchanged with culture medium. On day 3, 24-well plates were coated with 12.5 μg/ml recombinant retronectin (Takara Biotech, Japan) for 2 h at room temperature, blocked with 2% bovine serum albumin (Roth, Germany) for 30 min at 37° C. and washed with PBS. Concurrently, supernatant of the Plat-E cultures was harvested and replaced with fresh T cell medium. The harvested Plat-E supernatant was passed through a 40 μm filter (Milipore, USA) and 1 ml was distributed in each well of the 24 well plate, spinoculated for 2 hours at 4° C. and subseqeuntly removed. 10⁶ T cells were seeded in each well of the 24 well plate in 1 ml T cell medium supplemented with 10 U IL-2 and 400,000 anti-CD3 and anti-CD28 beads (Invitrogen, Germany) per well, and spinoculated at 800 g for 30 min at 32° C. On day 4, Plat E supernatant was again harvested and filtered. 1 ml was added to each well of the 24-well plate and spinoculated at 800 g for 90 min at 32° C. Cells were subsequently incubated for 6 additional hours at 37° C., after which the Plat-E supernatant was replaced with T cell medium. On day 5, the T cells were harvested, counted and reseeded at 10⁶ cells/ml density in T cell medium supplemented with 10 ng human IL-15 per ml (Peprotech, Germany). T cells were maintained at this density until day 10 when cell analysis or functional assays were performed.

### Tumor Cell Lines

The murine pancreatic cell line Panc02 was acquired from ATCC. The Panc02-OVA cell line was generated by modifying Panc02 cells with retroviruses to express the chicken derived Ovalbumin antigen (OVA). Both Panc02 and Panc02-OVA have been previously described, e.g., in Jacobs et al., Int J Cancer 128(2011), 897-907.

All tumor lines were maintained in DMEM supplemented with 10% FCS, L-glutamine, and Pen/Strep (Gibco), and used for experiments when in exponential growth phase. Transduced tumor cell lines were tested for OVA expression by flow cytometry using anti-mouse H-2Kb (clone 25-D1.16, ebioscience, Germany). Transduced tumor lines were also tested against antigen specific T cell for IFN-γ release, measured by ELISA. All cell lines used in experiments described herein were regularly checked for mycoplasma species with the commercial testing kit MycoAlert (Lonza).

### Migration Assay and Activity

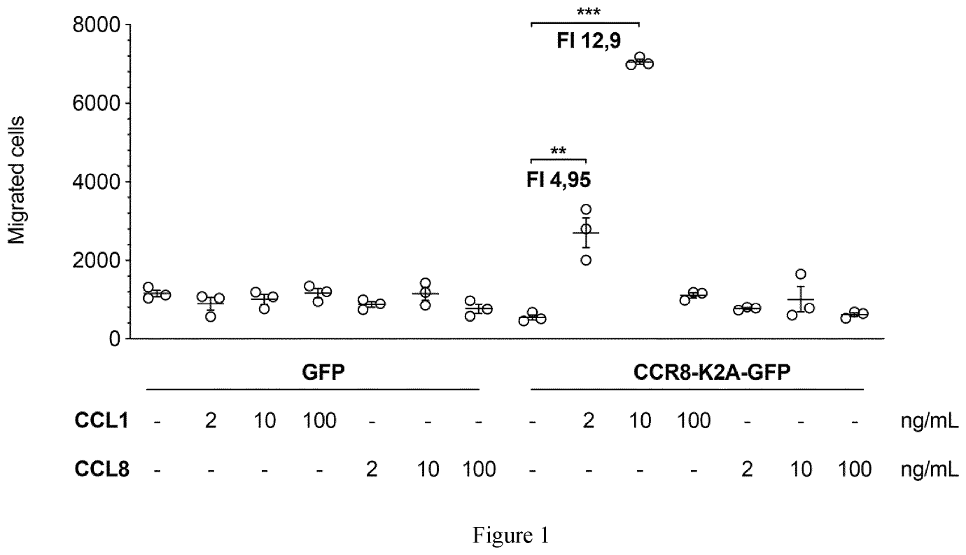

CCR8-transduced OT-1 T cell and GFP-transduced control OT-1 T cells were compared for CCL1 induced chemotactic activity (i.e., the ability to migrate towards a CCL1 gradient) using 96 well transwell plates (Corning). Migration medium (0.5% BSA in RPMI medium) was used with or without recombinant CCL1 (Biolegend, USA) or CCL8 (Peprotech, Germany) at dilutions of 2, 10 or 100 mg/ml in the lower chamber. 1×10⁶ T cells were placed onto a 3 μm pore membrane in the upper chamber of each well. After 3 h incubation at 37° C. the migrated T cells from the lower chamber were quantified by flow cytometry. As shown in FIG. 1, CCR8-transduced T cells specifically and dose dependently migrated towards CCL1 but not CCL8. No migration to either chemokine was seen with T cells transduced with GFP alone. P-values are depicted in the Figure, ** indicates p<0.01 and *** p<0.001.

### Tumor Model and Activity

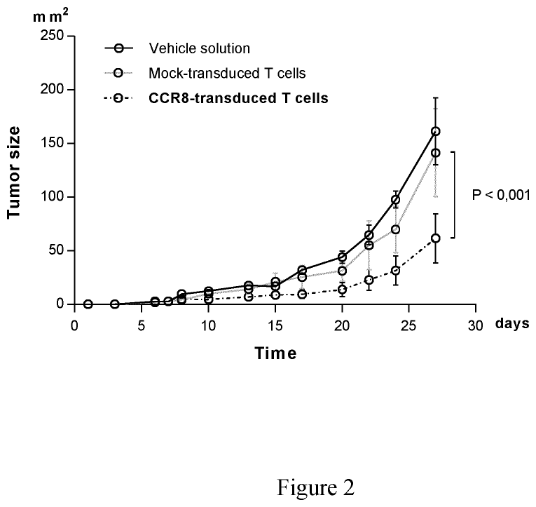

2×10⁶ Panc02-OVA cells in 100 μl PBS were injected subcutaneously into the flanks of female C57BL/6 mice. Animals were randomized into treatment groups (n=5 per group) according to tumor volumes. Once the tumor volumes had reached at least 30 mm³, ACT was initiated by the injection of 10⁷ T cells i.v. via the tail vein. Tumor volumes were measured before ACT and every second to third day after treatment start, and calculated as V=(length×width2)/2. As shown in FIG. 2, treatment of established Panc02-OVA models with antigen-specific T cells transduced with CCR8 lead to a superior anti-tumor activity compared to mock transduced T cells. In combination with the results of the migration studies (FIG. 1), the enhanced therapeutic activity is suggested to be due to the improved chemotactic and infiltration activity of the CCR8 expressing cells.

### Example 2: CCL1 Expression of Immune Cells

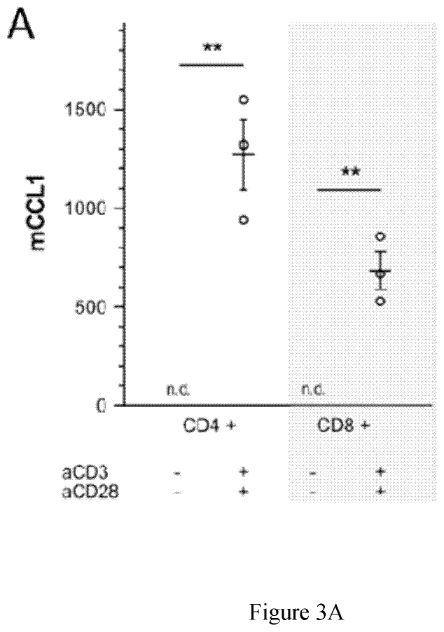

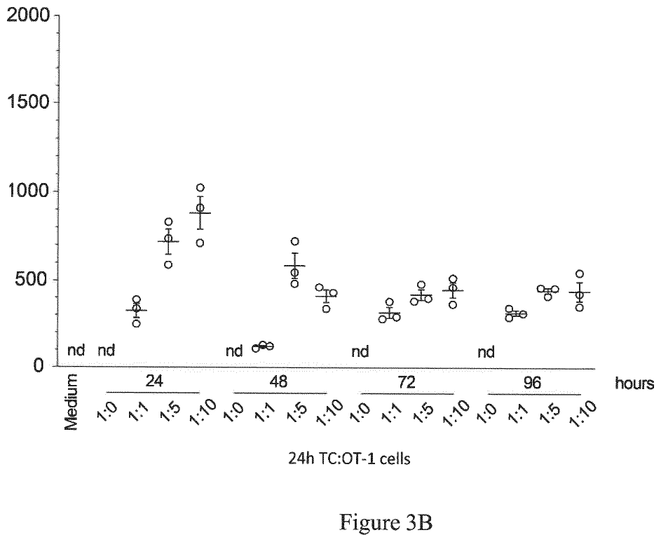

OT-1 T cells were assessed for CCL1 expression subsequent to activation by anti-CD3 and anti-CD28 antibodies (monoclonal antibody 145-2C11, eBioscience, cat #14-0031-86; and monoclonal antibody 37.51, functional grade, eBioscience, cat #16-0281-85; resepctively, both from Thermofischer Scientific, Germany). CD4+ and CD8+ cells were isolated using isolation kits available from Miltenyi Biotec, Germany, according to manufacturer's instrcutions. After isolation, cells were cultured with our without anti-CD3 and anti-CD28 antibodies. Supernatant was removed and tested for CCL1 by ELISA by a kit pursuant to manufacturer's instuctions (R&D Systems, Germany). As shown in FIG. 3A, on activation, both CD4+ and CD8+ T cells expressed CCL1. P-values are depicted in the Figure, ** indicates p<0.01.

Panc02-OVA cells (0.03×10⁶/well) were co-cultured with OT-1 T cells prepared as described in Example 1 at ratios of 1:0, 1:1, 1:5 and 1:10 in 96-well plates (flat bottom). Supernatants were harvested at 24, 48, 72 and 96 hours. CCL1 secretion was determined as described above. As shown in FIG. 3B, the antigen (OVA) recognition in the context of MEW on the surface of the tumor cells by the antigen-specific T cells induced the rapid expression/secretion of CCL1 by the T cells with the first 24 hours.

### Example 3: Expression of CCL1 in Panc02-OVA Tumor Bearing Mice

Expression of CCL1 in various organs of mice bearing the Panc02-OVA tumor model were analyzed over time and compared to control mice. The Panc02-OVA model was established in female C57BL/6 mice as described in Example 1. Organs and tumors were harvested one, two or three weeks after induction and frozen in liquid nitrogen to allow concrent processing of all samples. After determination of protein content by the Bradford method (Bio Rad, Munchen), CCL1 expression was measured by CCL1 ELISA kit pursuant to manufacturer's instructions (R&D Systems, Germany).

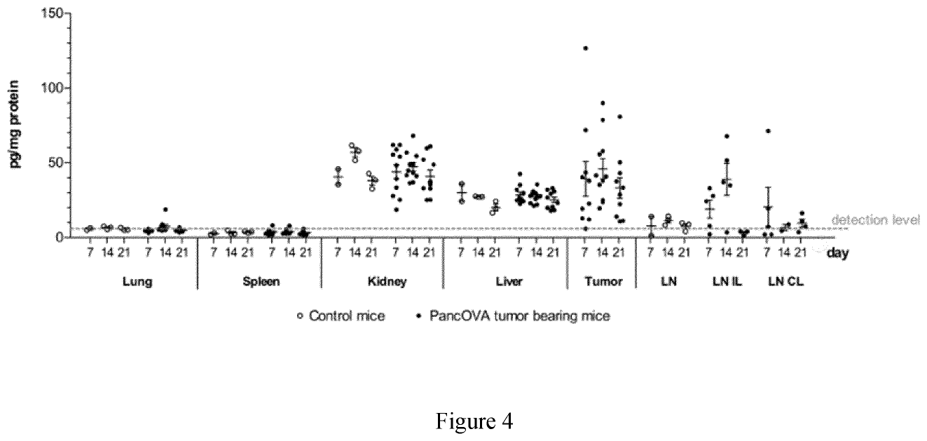

As shown in FIG. 4, CCL1 expression is not affected by disease state, i.e., the CCL1 is unchanged in the sampled organs of the tumor bearing mice as compared to control mice. However, CCL1 was detected at significant levels in tumor tissue, which is the primary site of CCL1 expression in the mouse model.

### Example 4: Therapeutic Effect of CCR8 Transduction on ACT Using Tumor-Antigen Specific T Cells in a Panc02-OVA Murine Cancer Model that Overexpresses CCL1

Primary murine OT-1 T cells were isolated and transduced according to the general procedures of Example 1. Cells were transduced with vectors encoding GFP (control) or CCR8-GFP. Panc02-OVA cells according to example 1 were further transduced according to the same methods to additionallyexpress CCL1 (Panc02-OVA-CCL1). Vectors comprising multiple cistrons were linked at the DNA level with a viral 2A sequence, which upon ribosomal translation will result in the expresion of two independent proteins.

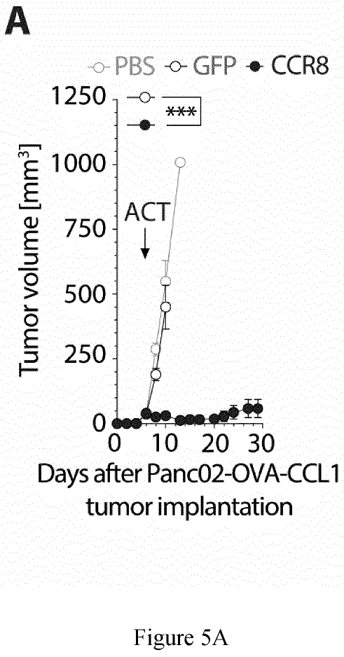

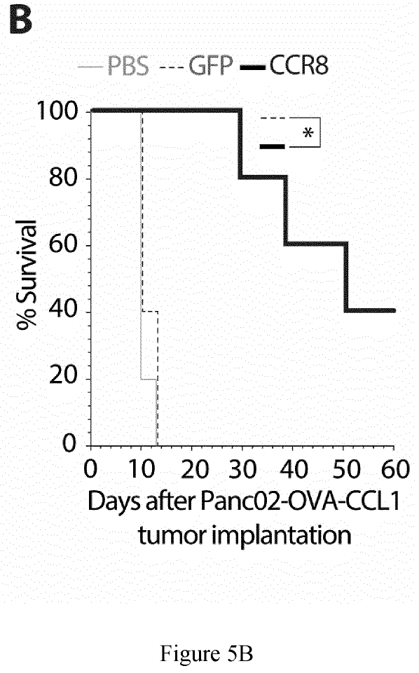

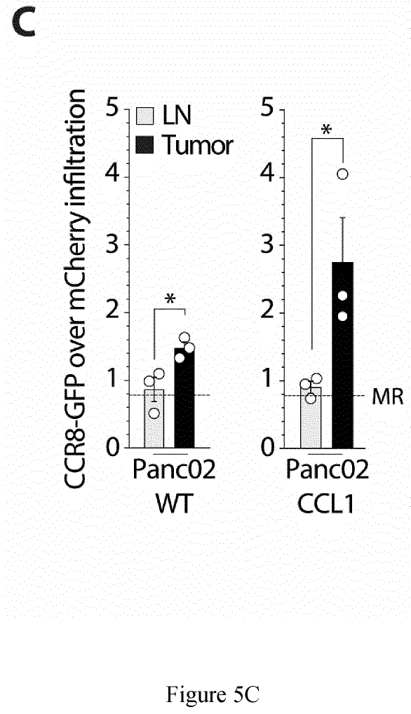

2×10⁶ Panc02-OVA-CCL1 cells in 100 μl PBS were injected subcutaneously into the flanks of female C57BL/6 mice. Animals were randomized into treatment groups (n=5 per group) according to tumor volumes. Once the tumor volumes had reached at least 30 mm³, ACT was initiated by the injection of 10⁷ T cells i. v. via the tail vein. Tumor volumes were measured before ACT and every second to third day after treatment start, and calculated as V=(length×width2)/2. As shown in FIGS. 5 A and B, treatment of established Panc02-OVA-CCL1 models with antigen-specific T cells transduced with CCR8 lead to a superior anti-tumor activity compared to control (GFP only) T cells. In combination with the results of the migration studies, the improved efficacy observed in Panc02-OVA-CCL1 model as compared with that in the Panc02-OVA model of Example 1 (FIG. 1) suggests that the improved effect is due to increased T cell migration in response to the increased expression of CCL1 in the tumor tissue.

To directly assess T cell migration and tumor infiltration, 2×10⁶ Panc02 or Panc02-CCL1 cells in 100 μl PBS were injected subcutaneously into the flanks of female C57BL/6 mice. Once the tumor volumes had reached at least 30 mm³, ACT was initiated by the injection of a mixture of 5×10⁶ OT-1, CCR8-GFP T cells and 5×10⁶ OT-1, mCherry (control) T cells i. v. via the tail vein that. Mice were sacrificed 3 days after ACT and organs were processed for T cell tracking using flow cytometry. As shown in FIG. 5C, tumor tissue showed specific increased infiltration of CCR8-GFP T cells compared to mCherry control T cells, whereas control organs such as lymph nodes did not show preferential infiltration. The effects were even more robust in the Panc02-CCL1 cell model, revealing that CCR8-mediated migration and infiltration is indeed responding to CCL1 expression.

### Example 5: Influence of CCR8 Transduction on T Cells Recmbinantly Expressing a Chimeric Antigen Receptor (CAR)

Primary murine T cells were isolated from wild type C57Bl/6 mice and transduced according to the general procedures of Example 1. Cells were transduced with vectors encoding mCherry (control), CCR8-GFP, anti-EpCAM CAR (“CAR47”)-mCherry, CAR47-CCR8, CCR8-CAR47, or left untransduced (the nucleotide sequence encoding the anti-EpCAM CAR is provided in SEQ ID NO:5). Vectors comprising multiple cistrons were linked at the DNA level with a viral 2A sequence, which upon ribosomal translation will result in the expression of two independent proteins.

Transduced T cells at a concentration of 2.5×10⁶ cells/ml were seeded in a 96 well plate (total volume 200 μl) and stimulated with either (i) anti-CD3 and anti-CD28 antibodies (at 1:1000 and 1:10000 dilution, respectively) or (ii) cocultured with Panc02-EpCAM tumor cells at a concentration of 1×10⁶ cells/ml (included in the 200 μl total volume within the well). Experiments were performed in triplicate with control wells having medium only, T cells only, or Panc02-EpCAM tumor cells only.

Activation of the transduced T cells was determined by IFN-γ release and/or cytotoxicity of the EpCAM expressing cells by LDH release (LDH Kit, Promega, Germany). After 24 hours culture/stimulation, the plates were centrifuged and supernatants carefully removed to avoid transferring cells. Supernatants were diluted 1:10, 1:200, or 1:500 and examined with murine IFN-γ ELISA kits (BD Biosciences) according to the manufacturer's instructions.

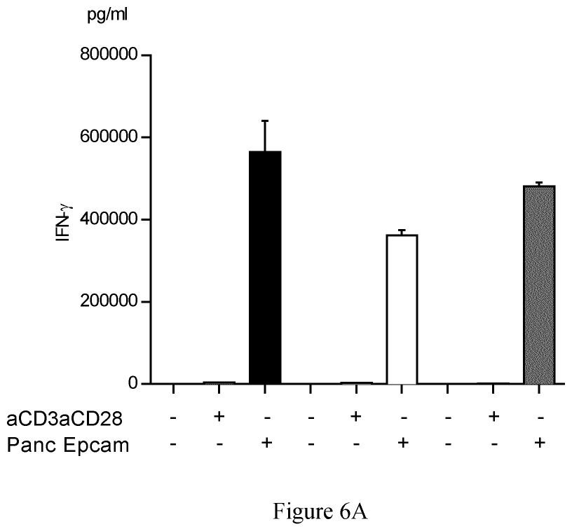

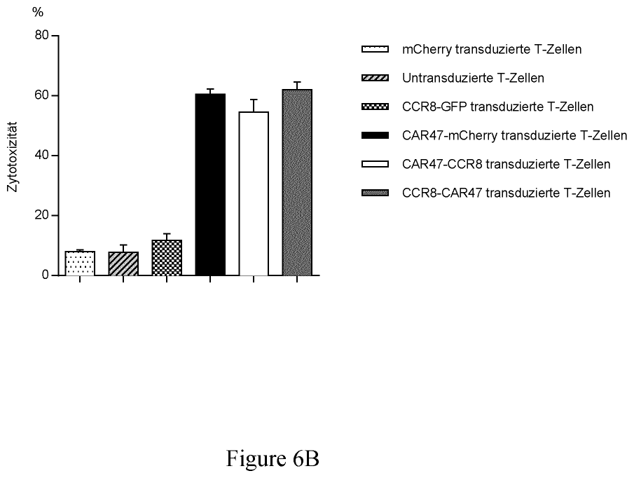

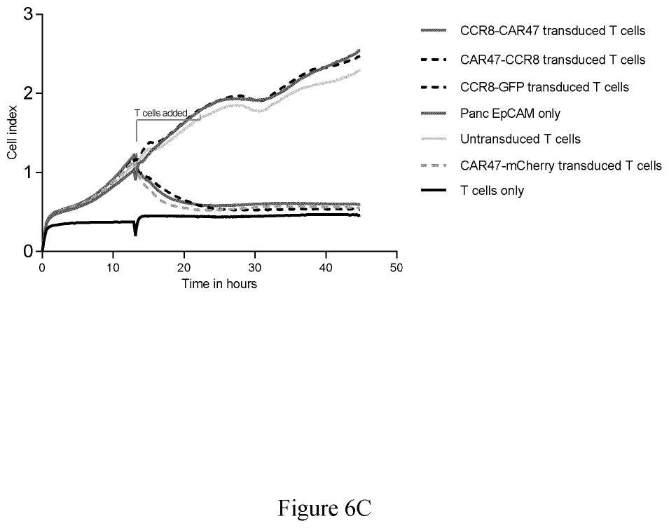

As demonstrated in FIG. 6A, as determined by IFN-γ release, co-culture with Panc02-EpCAM cells resulted in significantly improved activitation as compared with that achieved with the combinations of anti-CD3 and anti-CD28 antibodies, which activation was at the limit of detection. As demonstrated by FIG. 6B, cells transduced with CAR47 exhibited substantial cytotoxic activity against the EpCAM expressing cells, which cytoxicity was not affected by the co-transduction/co-expression of CCR8.

The cytotoxic activity of the transduced T cells was additionally monitored by a further assay. Panc02-EpCAM tumor cells were seeded in xcelligence plates (ACEA Biosciences, CA, USA) and cultured for 12 hours. Subsequently T cells were added. Cell index was monitored with the bundled software and pursuant to the manufacturer's specifications and instructions; FIG. 6C. Cell index is a massless measurement correlated with cell adhesion (and, thus, viability) to the xcelligence plate. Destruction of target cells is associated with a decrease in cell index, allowing real-time monitoring of cytolytic activity of, e.g., effector cells. Thus, FIG. 6C confirms that transduction with CAR47 imparts T cells with cytolytic activity against EpCAM expressing cells, which activity is, again, not affected by the co-transduction/co-expression of CCR8.

### Example 6: The Combination of CCR8 and Anti-Tumor Activity in Lymphocytes Leads to Therapeutic Improvement Over Anti-Tumor Activity Alone

A murine tumor model was established as described in Example 1, but using Panc02-EpCAM cells instead of the Panc02-OVA line. Tumors were established by s.c. injection of 2×10⁶ cells into the left flank of each C57/Bl/6 mouse.

Primary murine T cells from C57/Bl/6 mice were transduced with CCR8-CAR47 or CAR47-mCHerry according to Example 5. When tumors reached approximately 2×3 mm in size, the mice were injected i.v. with 10×10⁶ of either transduced cell type with control mice receiving PBS. Tumor size was measured at lest three times a week to monitor tumor growth and development.

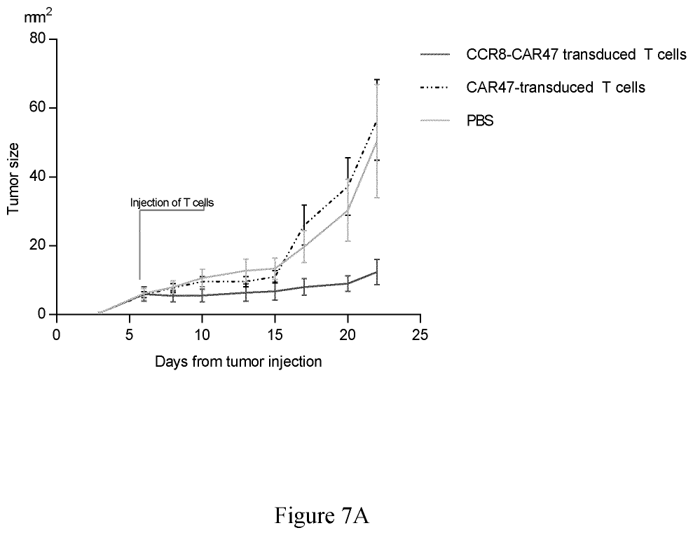

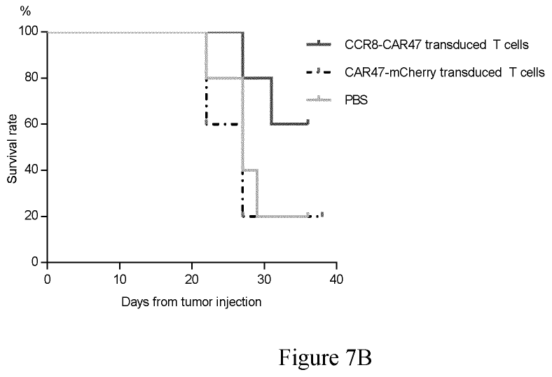

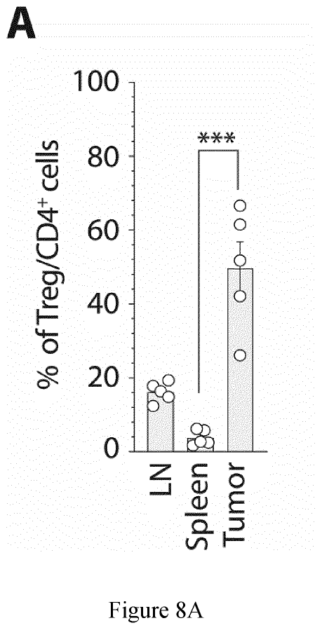

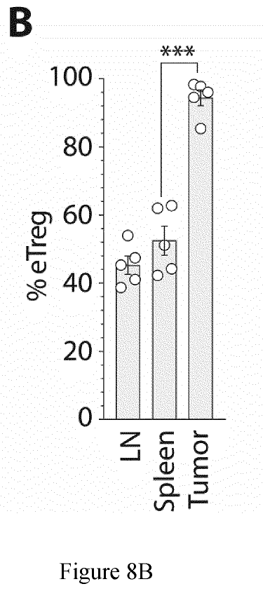

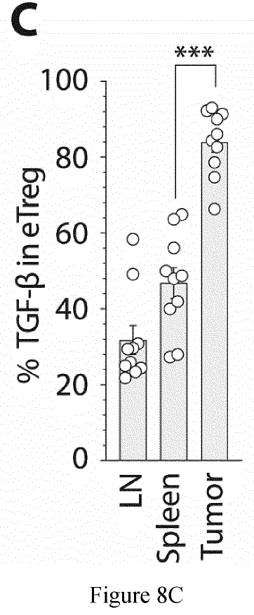

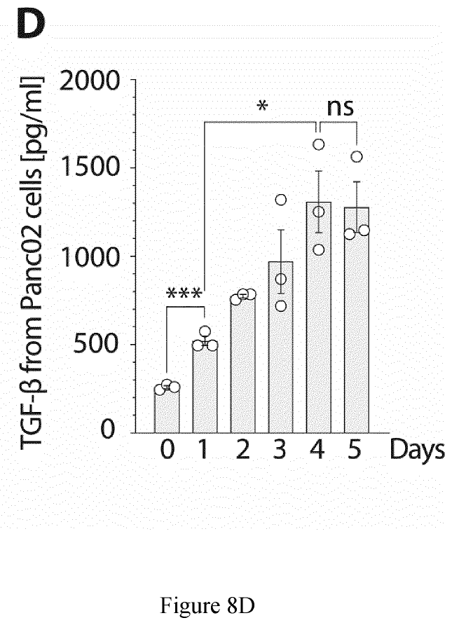

FIG. 7 demonstrates that cointroducing CCR8 together with a tumor-specific CAR enables CAR activity in a model otherwise resistant to CAR treatment, resulting in prolongation of survival and tumor rejection.

### Example 7: Panc02-OVA Tumors have Microenvironements Rich in Immunosuppressive Cells and Cytokines

A murine tumor model was established as described in Example 1 using the Panc02-OVA line. Tumors were established by s.c. injection of 2×10⁶ cells into the left flank of each C57/Bl/6 mouse. Once the tumors had reached 7×7 mm, the tumor bearing mice were sacrificed and their organs analysed by flow cytometry. Tumor mass, in comparison to lymph nodes and spleens had an increased ratio of regulatory T cells over CD4+ T cells (FIG. 8A). Furthermore, these regulatory T cells were predominantly of an effector subtype (FIG. 8B) and expressed in a high percentage TGF-β (FIG. 8C).

The expression of TGF-β in Panc02-OVA cells was examined my monitoring production in the supernatant in vitro. FIG. 8D demonstrates that Panc02-OVA tumor cells have the capacity to produce TGF-β in a time dependent manner.

### Example 8: Dominant-Negative TGF-β Receptor 2 can be Functionally Expressed in T Cells

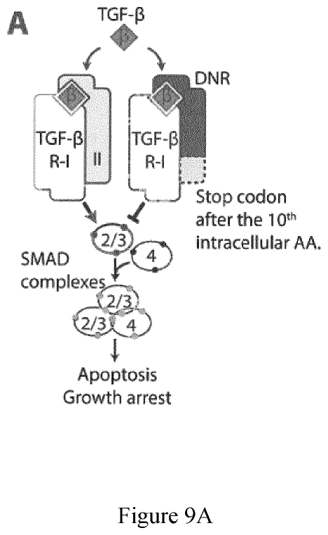

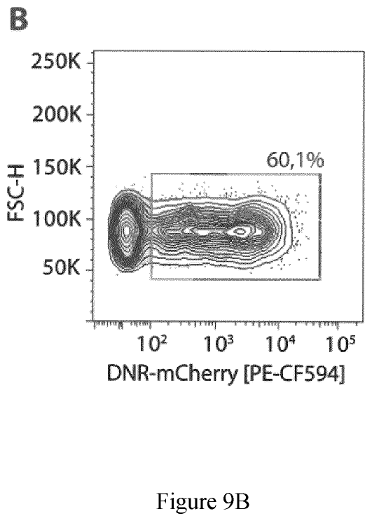

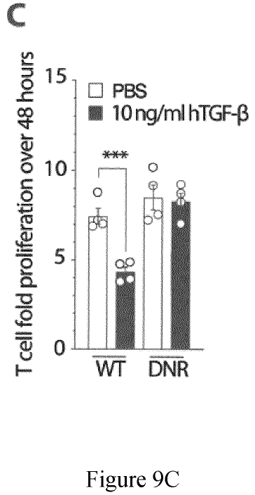

To investigate the role of TGF-β signaling in T cell regulation and potential impact on ACT, T cells were transduced to express Dominant-Negative TGF-β receptor 2 (DNR). The modified receptor, DNR, is known in the art (schematic provided in FIG. 9A); see, e.g. Siegel et al., P.N.A.S. 100(2003), 8430-8435; amino acid sequence encoded by SEQ ID NO:6. The lack of the endogenous intracellular domain enables DNR to act as a sink for TGF-β, protecting T cells from the suppressive effects of TGF-β, in particular, decreased proliferation and decreased cytotoxicity. T cells were transduced with DNR according to the methods of Example 1 and analyzed for its recombinant expression as shown in FIG. 9B. FIG. 9C shows the effects of DNR transduction/expression on the proliferation of T cells cultured with TGF-β (10 ng/ml during 24 hours) as compared to T cells prepared similarly but mock transduced. DNR transduced T cells cultured with TGF-β are able to proliferate as well as untransduced control that have been cultured without TGF-β.

### Example 9: Functionality of DNR Transduced T Cells In Vivo

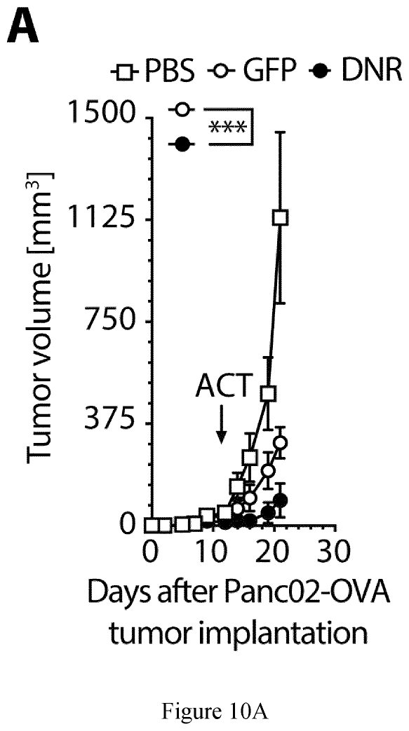

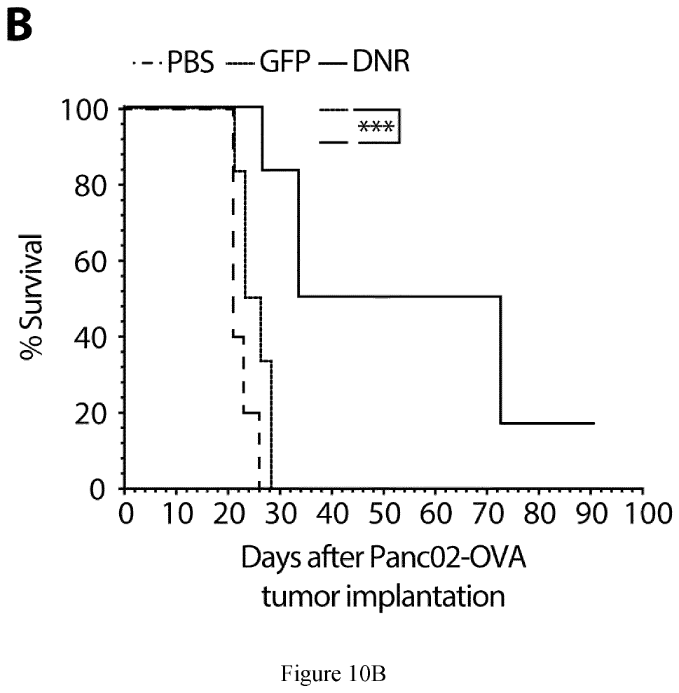

The Panc02-OVA in vivo model was established as in Example 1. Briefly, 2×10⁶ Panc02-OVA cells in 100 μl PBS were injected subcutaneously into the flanks of female C57BL/6 mice. Animals were randomized into treatment groups (n=5 per group) according to tumor volumes. Once the tumor volumes had reached at least 30 mm³, ACT was initiated by the injection of 10⁷ T cells i.v. via the tail vein. Treatment groups included those administered PBS or antigen-specific T cells (OT-1) that were transduced with DNR or GFP (mock/control). As shown in FIGS. 10 A and B, treatment of established Panc02-OVA models with antigen-specific T cells transduced with DNR leads to a superior anti-tumor activity as well as improved survival rates as compared to treatment with control T cells.

### Example 10: Functionality of the Combinatorial Therapy CCR8-DNR-CAR Transduced T Cells In Vivo

Primary murine T cells were isolated from wild type C57Bl/6 mice and transduced according to the general procedures of Example 1. Cells were transduced with vectors encoding anti-EpCAM CAR (CAR47)-mCherry, DNR-CAR47, CCR8-CAR47, or CCR8-DNR-CAR. Vectors comprising multiple cistrons were linked at the DNA level with a viral 2A sequence, which upon ribosomal translation will result in the expression of two independent proteins.

Panc02-EpCAM tumor cells were used as described in example 6. Tumors were established by s.c. injection of 2×10⁶ cells into the left flank of each C57/Bl/6 mouse. When tumors reached approximately 2×3 mm in size, the mice were injected i.v. with 10⁷ of either transduced cell type with control mice receiving PBS. Tumor size was measured at lest three times a week to monitor tumor growth and development.

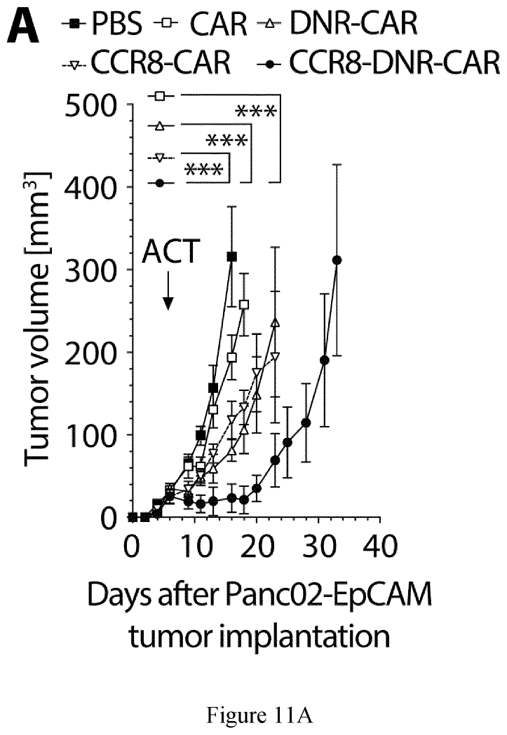

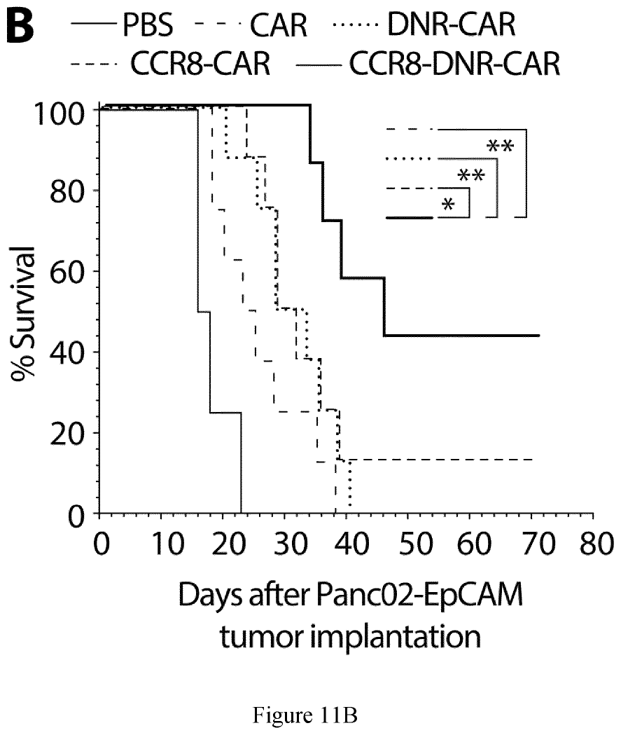

FIGS. 11 A and B demonstrate that the cointroduction of CCR8, DNR and CAR leads to the most potent anti-tumor activity of all the experimentalo condictions, resulting in prolongation of survival and tumor rejection.

### Example 11: Functionality of the Combinatorial Therapy CCR8-DNR-CAR Transduced T Cells In Vivo in a Xenograft Human Tumor Model

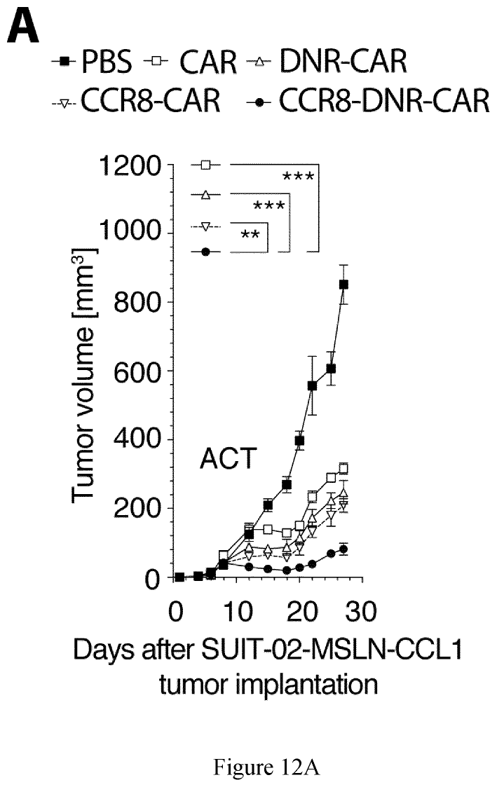

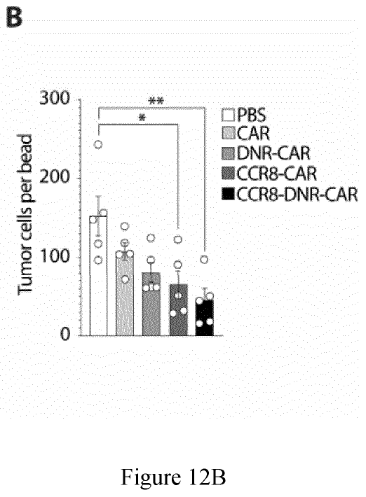

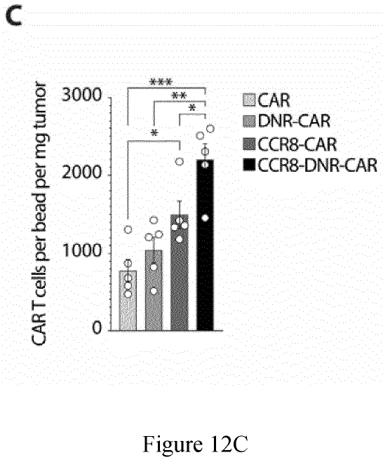

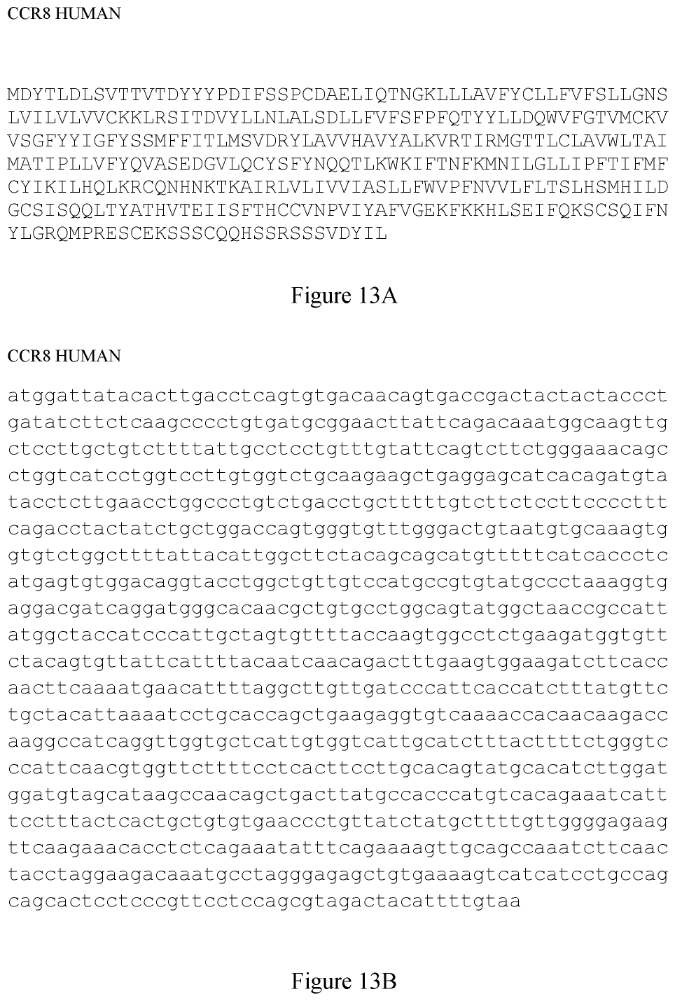

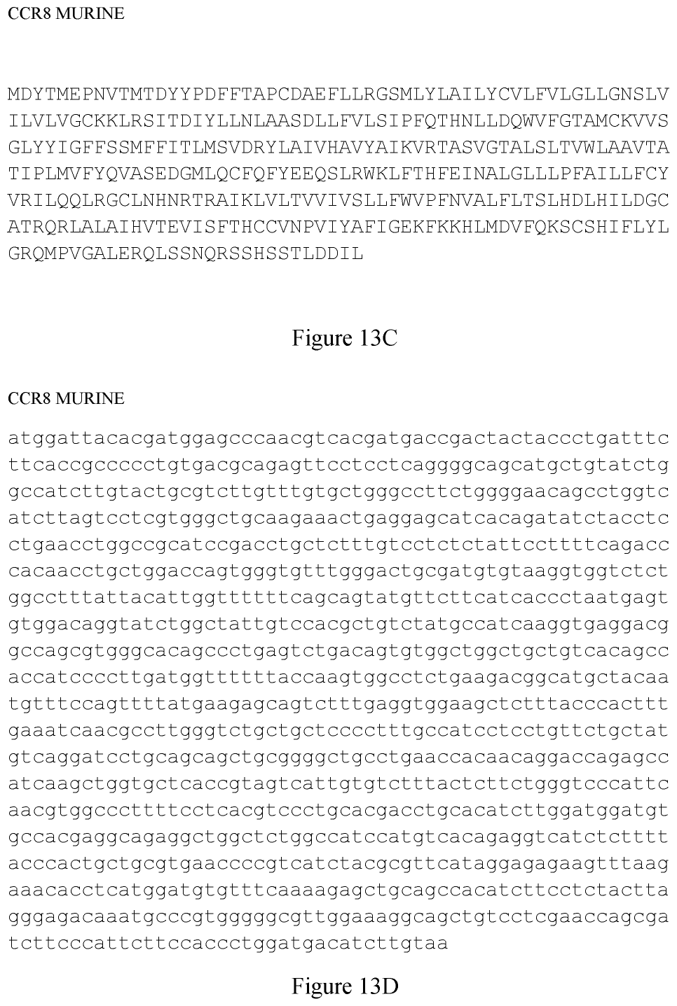

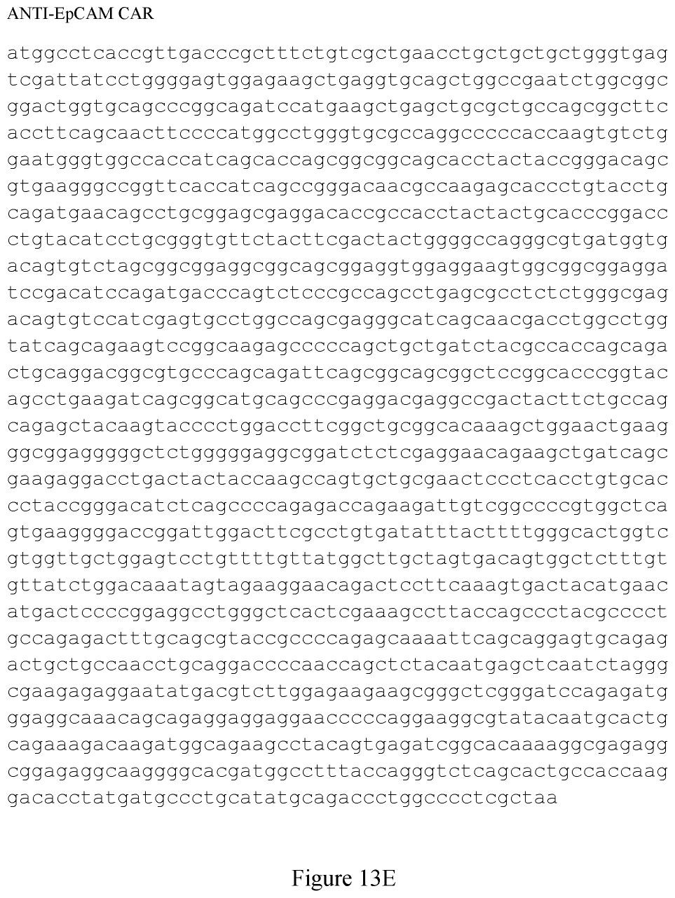

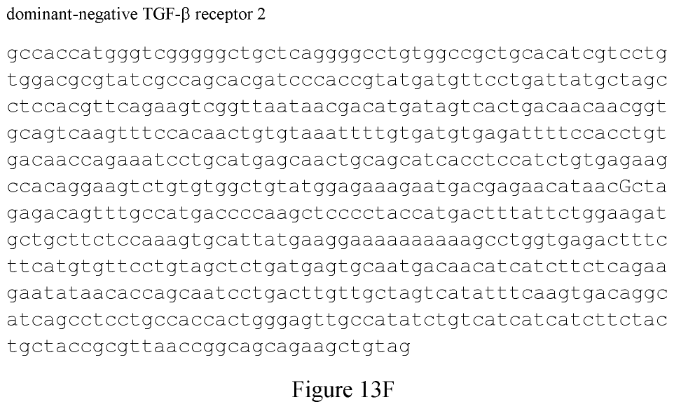

The above findings were further verified in a human pancreatic xenograft tumor model. Primary human T cells were differentiated using known protocols (e.g. Rapp et al., Oncoimmunology 5(2015), e1105428) with an anti-mesothelin (MSLN) CAR (Adusumilli et al., Science Translational Medicine 6 (Nov. 5, 2014), 261ra151) together with human CCR8, DNR, or both. The transduced T cells were assessed in a the human pancreatic xenograft tumor model using SUIT-2-MSLN-CCL1 tumors implanted in NOD-scid IL2rγnull (NSG) mice. CCR8-DNR-CAR outperformed single or double transduced T cells in controlling tumor-growth (FIG. 12A). CCR8-DNR-CAR T cell treatment efficacy was confirmed by a significant reduction of tumor cells compared to control conditions (FIG. 12B), and enhanced accumulation of transferred T cells at the tumor (FIG. 12C). This reiterated previous findings that the most pronounced reduction of tumor burden and highest expansion of T cell product were observed in CCR8-DNR-CAR T cell treated animals.

1. A primary human lymphocyte genetically engineered to express
(a) a chemokine receptor 8 polypeptide (CCR8) having the amino acid sequence of SEQ ID NO:1;
(b) a variant CCR8 polypeptide having an amino acid sequence at least 85% identical to SEQ ID NO:1 and further characterized by having CCR8 activity; or
(c) a fragment of the polypeptide of (a) or (b), wherein the fragment is characterized by having CCR8 activity.

2. The primary human lymphocyte according to claim 1 comprising
(a) an exogenous polynucleotide sequence encoding a polypeptide having the amino acid sequence of SEQ ID NO:1;
(b) a polynucleotide sequence encoding a variant CCR8 polypeptide having an amino acid sequence at least 85% identical to SEQ ID NO:1 and further characterized by having CCR8 activity;
(c) a polynucleotide sequence encoding a fragment of the encoded polypeptide of (a) or (b), wherein the fragment is characterized by having CCR8 activity;
(d) a polynucleotide comprising or consisting of the nucleic acid sequence SEQ ID NO:2; or
(e) a polynucleotide sequence having at least 85% identity to SEQ ID NO:2, which encodes a polypeptide having CCR8 activity.

3. The primary human lymphocyte according to claim 1, wherein said CCR8 activity is CCL1-induced chemotaxis of said lymphocyte or CCL1-induced binding to ICAM-1.

4. The primary human lymphocyte according to claim 1, which is a T cell or a NK cell.

5. The primary human lymphocyte according to claim 4 that is a T cell, wherein said T cell is a CD3+ T cell, a CD8+ T cell, a CD4+ T cell, a γδ T cell, an invariant T cell or a NK T cell.

6. The primary human lymphocyte according to claim 1, wherein said lymphocyte is non-alloreactive.

7. The primary human lymphocyte according to claim 6 that is a T cell, wherein said T cell comprises genetic modifications to reduce or eliminate expression of the T cell receptor (TCR) alpha or beta chain genes, or exhibits reduced or eliminated expression of the endogenous TCR.

8. The primary human lymphocyte according to claim 7 further genetically engineered to express a chimeric antigen receptor (CAR), an exogenous T cell receptor (TCR), or a modified cytokine receptor.

9. The primary human lymphocyte according to claim 8, wherein said lymphocyte is further genetically engineered to express a modified cytokine receptor that is dominant-negative TGF-β receptor 2 (DNR).

10. (canceled)

11. A method for the production of a lymphocyte genetically engineered to express a CCR8 polypeptide (SEQ ID NO:1), an amino acid sequence variant CCR8 polypeptide, or a fragment of either, comprising:
(a) introducing into the lymphocyte
(i) an exogenous polynucleotide encoding SEQ ID NO:1;
(ii) a polynucleotide encoding a polypeptide having an amino acid sequence at least 85% identical to SEQ ID NO:1 and which is further characterized in having CCR8 activity;
(iii) a polynucleotide encoding a fragment of the polypeptide encoded by the polynucleotide of (i) or (ii), which fragment is further characterized in having CCR8 activity;
(iv) a polynucleotide comprising or consisting of the nucleic acid sequence of SEQ ID NO:2; or
(v) a polynucleotide comprising or consisting of a nucleic acid sequence having at least 85% sequence identity to SEQ ID NO:2 that encodes a polypeptide characterized in having CCR8 activity.
(b) culturing the lymphocyte engineered according to (a) under conditions allowing the expression of the CCR8 polypeptide, amino acid sequence variant CCR8 polypeptide, or fragment of either; and
(c) recovering the engineered lymphocyte.

12. The method according to claim 11, wherein said CCR8 activity is CCL1-induced chemotaxis of said lymphocyte or CCL1-induced binding to ICAM-1.

13-14. (canceled)

15. The method according to claim 11, wherein said lymphocyte is a T cell or a NK cell.

16. The method according to claim 11, wherein said lymphocyte is a T cell that is a CD3+ T cell, a CD8+ T cell, a CD4+ T cell, a γδ T cell, an invariant T cell or a NK T cell.

17. The method according to claim 11, wherein said lymphocyte is non-alloreactive or is further genetically engineered so that it is non-alloreactive.

18. The method according to claim 11, wherein said lymphocyte is further genetically engineered to express a chimeric antigen receptor (CAR), an exogenous T cell receptor (TCR), or a modified cytokine receptor.

19. The method according to claim 18, wherein said lymphocyte is further genetically engineered to express a modified cytokine receptor that is dominant-negative TGF-β receptor 2 (DNR).

20-21. (canceled)

22. The method according to claim 11, wherein the lymphocyte is expanded after said genetic engineering by exposure to one or more of
(a) anti-CD3 antibodies;
(b) anti-CD28 antibodies; and
(c) one or more cytokines.

23-29. (canceled)

30. A pharmaceutical composition comprising the genetically engineered lymphocyte according to claim 1.

31-32. (canceled)

33. A method of treating a cancer comprising administering to a subject in need thereof the primary human lymphocyte according to claim 1, wherein said cancer is characterized by the expression of CCL within the tumor parenchyma or characterized by comprising CCL1 expressing tumor resident immune cells.

34. The method according to claim 33, wherein said primary human lymphocyte is allogenic to said subject.
